(19) United States
(12) Patent Application Publication
(10) Pub. No.: US 2003/0149000 A1
(43) Pub. Date: Aug. 7, 2003

(54) Method for the treatment of disorders associated with apoptosis using N-heterocyclic glyoxylamide compounds

(75) Inventors: Tatsurou Yagami, Siga (JP); Nobuo Takasu, Osaka (JP)

Correspondence Address: FOLEY AND LARDNER, SUITE 500, 3000 K STREET NW, WASHINGTON, DC, 20007, US

(73) Assignee: Shionogi & Co., Ltd.

(21) Appl. No.: 10/219,931

(22) Filed: Aug. 16, 2002

Related U.S. Application Data
(63) Continuation of application No. 09/530,781, filed on May 5, 2000.
Application No. 09/530,781 is a continuation-in-part of application No. PCT/JP98/05042, filed on Nov. 10, 1998.

(51) Int. Cl. A61K 31/675; A61K 31/4745; A61K 31/405

(52) U.S. Cl. 514/79; 514/300; 514/419

(57) ABSTRACT

A method or composition is disclosed for the treatment of disorders associated with apoptosis using N-heterocyclic glyoxylamide compounds.

This invention relates to a method for the treatment of disorders associated with apoptosis using N-heterocyclic glyoxylamide compounds, a use of N-heterocyclic glyoxylamide compounds for the treatment of disorders associated with apoptosis and a composition for the treatment of disorders associated with apoptosis comprising N-heterocyclic glyoxylamide compounds.

### BACKGROUND OF THE INVENTION

In multicellular organisms, homeostasis is maintained through a balance between cell proliferation and cell death. Cell death is roughly classified into necrosis and apoptosis. Apoptosis is observed in a physiological ontogeny, or an appearance of disorders or pharmaceutics effects, and it has been thought to occur on the basis of activation of the nature program in individual cell, which differs from necrosis. Apoptosis and necrosis are different from each other in terms of that apoptosis is associated with RNA synthesis and protein synthesis, while necrosis is not.

Although apoptosis-inducing stimulus and the mechanism thereof are various, its morphologic features are common. The first morphologic change is a formation of condensation of chromatin, which is almost associated with DNA fragmentation. Then the condensation is observed, it appears that the compaction of cytoplasm occurs, and cell itself forms cell fragments called apoptotic bodies. The formed apoptotic bodies are quickly phagocytosed and disintegrated by adjacent cells or macrophages, and the like so as to lead apoptosis.

Brain Research 693 (1995) 101-111 and THE JOURNAL BIOLOGICAL CHEMISTRY Vol. 271, No.51, (1996) 32722-32728 disclose that secretory phospholipase A2 (sPLA2) has neurotoxicity, but it does not disclose that the neurotoxicity therein is associated with apoptosis. Brain Research 651 (1994) 353-356 discloses that group II PLA2 is expressed in rat brain after severe forebrain ischemia, but it does not disclose any relationship between group II PLA2 and neuronal death. Brain Research 752 (1997) 203-208 discloses that a phospholipase A2 inhibitor, quinacrine, reduces infarct-size in rats after transient middle cerebral artery occlusion, but it does not disclose that the infarction therein is associated with apoptosis. WO 96/40982 discloses that a PLA2 inhibitor is useful in the treatment of neurodegenerative diseases, but it does not disclose that a secretory PLA2 inhibitor such as compounds used in the present invention can be used to treat such diseases. U.S. Pat. No. 5,478,857 discloses that a PLA2 inhibitor is useful in the treatment of Alzheimer's diseases, but it does not disclose that a secretory PLA2 inhibitor such as compounds used in the present invention can be used to treat such diseases.

### SUMMARY OF THE INVENTION

It is an object of this invention to provide a method of treatment of a mammal, including a human, currently afflicted with disorders associated with apoptosis, said method comprising administering to said mammal a therapeutically effective amount of an N-heterocyclic glyoxylamide compound.

It is also an object of this invention to use an N-heterocyclic glyoxylamide compound for the manufacture of a medicament for treatment of a mammal, including a human, currently afflicted with disorders associated with apoptosis.

It is also an object of this invention to provide a composition for treatment of disorders associated with apoptosis, said composition comprising a therapeutically effective amount of an N-heterocyclic glyoxylamide compound.

The present invention is considered to be useful for disorders associated with apoptosis, in more detail, chronic diseases such as Alzheimer's disease, a number of scleroma, ataxia, talangiectasia, prion-induced neuronal cell death, and the like, or acute diseases such as stroke, and the like.

### TREATMENT METHODS

General Aspects of the Method:

It will be apparent to those skilled in the art that a compound of the present invention can be co-administered with other therapeutic or prophylactic agents and/or medicaments that are not medically incompatible therewith.

The regimen for treatment may stretch over many months or years so oral dosing is preferred for patient convenience and tolerance. With oral dosing, one to three oral doses per day, each from about 0.01 to about 50 mg/kg of body weight are used with preferred doses being from about 0.04 to about 5.0 mg/kg.

The specific dose of N-heterocyclic glyoxylamide compound administered according to this invention to obtain therapeutic or prophylactic effects will, of course, be determined by the particular circumstances surrounding the case, including, for example, the compound administered, the route of administration, the size and age of the patient, the severity of disorders associated with apoptosis, and the condition being treated. Typical daily doses will contain a non-toxic dosage level of from about 0.01 mg/kg to about 50 mg/kg of body weight of an active compound of this invention.

Method of Administration

The N-heterocyclic glyoxylamide compounds are most often used in the method of the invention in the form of pharmaceutical formulation, as described infra. Other forms of administration may be used in both human and veterinary contexts. Such alternative forms include the use of suppositories, transdermal patches, and compositions for buccal or nasal administration, for example lozenges, nose drops, an aerosol spray, or transdermal patch.

### COMPOUNDS USED IN TREATMENT METHOD OF THE DISORDERS ASSOCIATED WITH APOPTOSIS

The method for treating subjects for the occurrence or prevention of disorders associated with apoptosis comprises administering an effective amount of an N-heterocyclic glyoxylamide compound. Suitable 1H-indole-3-glyoxylamide compounds for the practice of the method of treating and preventing disorders associated with apoptosis as taught herein are those described in European Patent Application No. 95302166.4, Publication No. 0 675 110 (publ., Oct. 4, 1995). Suitable 1H-indole-3-glyoxylamide compounds are also those disclosed in U.S. patent application Ser. No. 08/469,954 filed Jun. 6, 1995, the disclosure of which is incorporated herein by reference. Formulations containing these 1H-indole-3-glyoxylamide compounds and methods of making them are also fully described in European Patent Office Publication European Patent Application No. 95302166.4 and U.S. patent application Ser. No 08/469,954. Suitable indolizine compounds are disclosed in WO 9603383 (Publ., Feb. 8, 1996).

Definitions:

The words, “acid linker” refers to a divalent linking group symbolized as, -(Lₐ)- or (La′)-, which has the function of joining the 4 or 5 position of the indole nucleus or the 7 or 8 position of the indolizine nucleus to an acidic group in the general relationship:

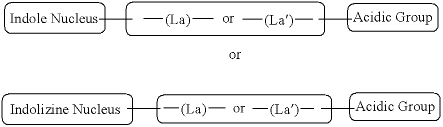

The words, “acid linker length”, refer to the number of atoms (excluding hydrogen) in the shortest chain of the linking group -(Lₐ)- or (La′)- that connects the 4 or 5 position of the indole nucleus or the 7 or 8 position of the indolizine nucleus with the acidic group.

The word “acidic group” is selected from -5-tetrazolyl, —SO₃H,

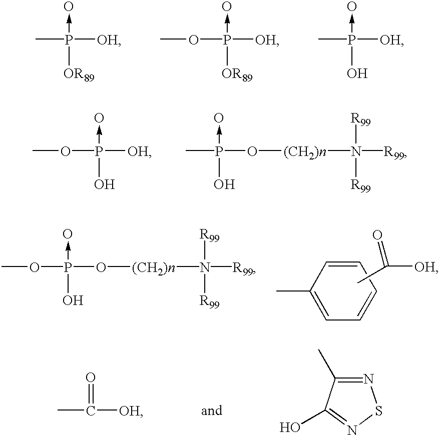

where

n is from 1 to 8, R₈₉ is a metal or C₁-C₁₀ alkyl, and R₉₉ is hydrogen or C₁-C₁₀ alkyl.

Preferred compounds for use in the method or composition of the invention are those having the general formula (I) or a pharmaceutically acceptable salt, solvate or prodrug derivative thereof;

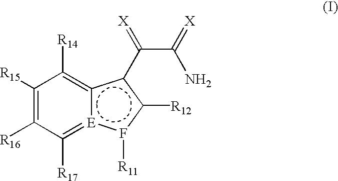

wherein

E and F are differently C or N;

--- is presence or absence of a double bond;

each X is independently oxygen or sulfur;

R₁₁ is selected from groups (a), (b) and (c) where;

(a) is C₇-C₂₀ alkyl, C₇-C₂₀ alkenyl, C₇-C₂₀ alkynyl; or carbocyclic radical selected from the group cycloalkyl, cycloalkenyl, phenyl, naphthyl, norbornanyl, bicycloheptadienyl, tolyl, xylyl, indenyl, stilbenyl, terphenylyl, diphenylethylenyl, phenyl-cyclohexenyl, acenaphthylenyl, and anthryl, biphenyl, bibenzylyl and related bibenzylyl homologues represented by the formula (bb),

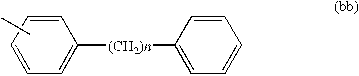

where

n is a number from 1 to 8; or

(b) is a member of (a) substituted with one or more independently selected non-interfering substituents selected from the group consisting of C₁-C₆ alkyl, C₂-C₆ alkenyl, C₂-C₆ alkynyl, C₇-C₁₂ aralkyl, C₇-C₁₂ alkaryl, C₃-C₈ cycloalkyl, C₃-C₈ cycloalkenyl, phenyl, tolyl, xylyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₁-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ—O—(C₁-C₆ alkyl), benzyloxy, phenoxy, phenylthio, —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl; where n is from 1 to 8;

(c) is the group -(L₁)-R₈₁; where, -(L₁)- is a divalent linking group having the formula;

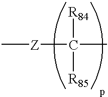

where,

R₈₄ and R₈₅ are each independently selected from hydrogen, C₁-C₁₀ alkyl, carboxyl, carbalkoxy, or halo;

p is 1 to 5,

Z is a bond, —(CH₂)—, —O—, —N(C₁-C₁₀ alkyl)-, —NH—, or —S—; and where R₈₁ is a group selected from (a) or (b);

R₁₂ is hydrogen, halo, C₁-C₃ alkyl, C₃-C₄ cycloalkyl, C₃-C₄ cycloalkenyl, —O—(C₁-C₂ alkyl), or —S—(C₁-C₂ alkyl);

R₁₄ is hydrogen or a group, -(Lₐ)-(acidic group) wherein -(Lₐ)- is represented by the formula;

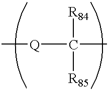

where

Q is selected from the group —(CH₂)—, —O—, —NH—, and —S—, and R₈₄ and R₈₅ are each independently selected from hydrogen, C₁-C₁₀ alkyl, aryl, C₁-C₁₀ alkaryl, C₁-C₁₀ aralkyl, and halo;

R₁₅ is hydrogen or a group, -(La′)-(acidic group) wherein -(La′)- is is represented by the formula;

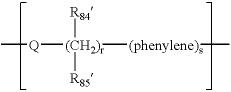

where

r is a number from 1 to 7, s is 0 or 1, and Q is selected from the group —(CH₂)—, —O—, —NH—, and —S—, and R_{84′} and R_{85′} are each independently selected from hydrogen, C₁-C₁₀ alkyl, aryl, C₁-C₁₀ alkaryl, C₁-C₁₀ aralkyl, carboxy, carbalkoxy, and halo; provided that at least one of R₁₄ or R₁₅ must be the group, -(La)-(acidic group) or -(La′)-(acidic group);

R₁₆ is hydrogen, carboxyl or ester thereof;

R₁₇ is selected from hydrogen, non-interfering substituents, selected from the group consisting of C₁-C₆ alkyl, C₂-C₆ alkenyl, C₂-C₆ alkynyl, C₇-C₁₂ aralkyl, C₇-C₁₂ alkaryl, C₃-C₈ cycloalkyl, C₃-C₈ cycloalkenyl, phenyl, tolyl, xylyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ—O—(C₁-C₆ alkyl), benzyloxy, phenoxy, phenylthio, —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl; where n is from 1 to 8.

A preferred class of compounds for the method or composition of the invention are compounds represented by the formula (II):

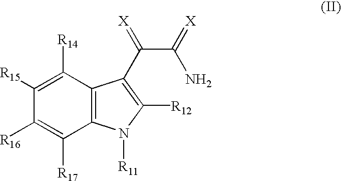

wherein

X, R₁₁, R₁₂, R₁₄, R₁₅, R₁₆ and R₁₇ are as defined above.

An alternatively preferred class of compounds for the method or composition of the invention are compounds represented by the formula (III):

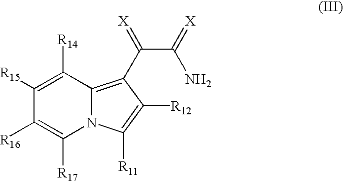

wherein

X, R₁₁, R₁₂, R₁₄, R₁₅, R₁₆ and R₁₇ are as defined above.

A further preferred class of compounds for the method or composition of the invention are the compounds represented by the formula (II) or (III) where both X's are oxygen, only one of R₁₄ or R₁₅ is -(Lₐ)-(acidic group) or -(La′)-(acidic group ), and the acidic group is carboxyl.

Specific preferred compounds and all pharmaceutically acceptable salts, solvates and prodrug derivatives thereof which are useful in the method or composition of the invention include the following:

(A) [[3-(2-Amino-1,2-dioxoethyl)-2-methyl-1-(phenylmethyl)-1H-indol-4-yl]oxy]acetic acid,

(B) dl-2-[[3-(2-Amino-1,2-dioxoethyl)-2-methyl-1-(phenylmethyl)-1H-indol-4-yl]oxy]propanoic acid,

(C) [[3-(2-Amino-1,2-dioxoethyl)-1-([1,1′-biphenyl]-2-ylmethyl)-2-methyl-1H-indol-4-yl]oxy]acetic acid,

(D) [[3-(2-Amino-1,2-dioxoethyl)-1-([1,1′-biphenyl]-3-ylmethyl)-2-methyl-1H-indol-4-yl]oxy]acetic acid,

(E) [[3-(2-Amino-1,2-dioxoethyl)-1-([1,1′-biphenyl]-4-ylmethyl)-2-methyl-1H-indol-4-yl]oxy]acetic acid,

(F) [[3-(2-Amino-1,2-dioxoethyl)-1-[(2,6-dichlorophenyl)methyl]-2-methyl-1H-indol-4-yl]oxy]acetic acid,

(G) [[3-(2-Amino-1,2-dioxoethyl)-1-[(4-fluorophenyl)methyl]-2-methyl-1H-indol-4-yl]oxy]acetic acid,

(H) [[3-(2-Amino-1,2-dioxoethyl)-2-methyl-1-[(1-naphthalenyl)methyl]-1H-indol-4-yl]oxy]acetic acid,

(I) [[3-(2-Amino-1,2-dioxoethyl)-2-ethyl-1-(phenylmethyl)-1H-indol-4-yl]oxy]acetic acid,

(I′) [[3-(2-Amino-1,2-dioxoethyl)-2-ethyl-1-(phenylmethyl)-1H-indol-4-yl]oxy]acetic acid methyl ester

(J) [[3-(2-Amino-1,2-dioxoethyl)-1-[(3-chlorophenyl)methyl]-2-ethyl-1H-indol-4-yl]oxy]acetic acid,

(K) [[3-(2-Amino-1,2-dioxoethyl)-1-([1,1′-biphenyl]-2-ylmethyl)-2-ethyl-1H-indol-4-yl]oxy]acetic acid,

(L) [[3-(2-amino-1,2-dioxoethyl)-1-([1,1′-biphenyl]-2-ylmethyl)-2-propyl-1H-indol-4-yl]oxy]acetic acid,

(M) [[3-(2-Amino-1,2-dioxoethyl)-2-cyclopropyl-1-(phenylmethyl)-1H-indol-4-yl]oxy]acetic acid,

(N) [[3-(2-Amino-1,2-dioxoethyl)-1-([1,1′-biphenyl]-2-ylmethyl)-2-cyclopropyl-1H-indol-4-yl]oxy]acetic acid,

(O) 4-[[3-(2-Amino-1,2-dioxoethyl)-2-ethyl-1-(phenylmethyl)-1H-indol-5-yl]oxy]butanoic acid,

(P) mixtures of (A) through (O) in any combination,

(Q) (8-(Carbomethoxymethyloxy)-2-ethyl-3-(o-phenylbenzyl)indolizin-1-yl)glyoxylamide,

(R) (3-Benzyl-8-(carbethoxymethyloxy)-2-ethylindolizin-1-yl)glyoxylamide,

(S) (8-(Carbethoxymethyloxy)-2-ethyl-3-(o-phenylbenzyl)indolizin-1-yl)glyoxylamide,

(T) (3-Benzyl-8-(carbethoxymethyloxy)-2-methylindolizin-1-yl)glyoxylamide,

(U) (8-(Carbethoxymethyloxy)-3-(m-chlorobenzyl)-2-ethylindolizin-1-yl)glyoxylamide,

(V) (8-Carbethoxymethyloxy-2-ethyl-3-(1-naphthylmethyl)indolizin-1-yl)glyoxylamide,

(W) (3-Benzyl-8-(t-butoxycarbonylmethyloxy)-2-ethylindolizin-1-yl)glyoxylamide,

(X) (8-(Carbmethoxymethyloxy)-2-ethyl-3-(m-trifluoromethylbenzyl)indolizin-1-yl)glyoxylamide,

(Y) (8-(Carbmethoxymethyloxy)-2-cyclopropyl-3-(o-phenylbenzyl)indolizin-1-yl)glyoxylamide,

(Z) (3-Benzyl-8-(carboxymethyloxy)-2-ethylindolizin-1-yl)glyoxylamide,

(AA) (8-(Carboxymethyloxy)-2-ethyl-3-(o-phenylbenzyl)indolizin-1-yl)glyoxylamide,

(AA′) (8-Carbomethoxymethyloxy)-2-ethyl-3-(o-phenylbenzyl)indolizin-1-yl)glyoxylamide,

(BB) (3-Benzyl-8-(carboxymethyloxy)-2-methylindolizin-1-yl)glyoxylamide,

(CC) (8-(Carboxymethyloxy)-3-(m-chlorobenzyl)-2-ethylindolizin-1-yl)glyoxylamide,

(DD) (8-(Carboxymethyloxy)-2-ethyl-3-(m-trifluoromethylbenzyl)indolizin-1-yl)glyoxylamide,

(EE) (8-Carboxymethyloxy-2-ethyl-3-(1-naphthylmethyl)indolizin-1-yl)glyoxylamide,

(FF) (8-(Carboxymethyloxy)-2-cyclopropyl-3-(o-phenylbenzyl)indolizin-1-yl) glyoxylamide,

(GG) mixtures of (Q) through (FF) in any combination,

(HH) [[3-(2-Amino-1,2-dioxoethyl)-6-carboxyl-2-ethyl-1-benzyl-1H-indol-4-yl]oxy]acetic acid,

(II) [[3-(2-Amino-1,2-dioxoethyl)-6-methoxycarbonyl-2-ethyl-1-benzyl-1H-indol-4-yl]oxy]acetic acid,

(JJ) [[3-(2-Amino-1,2-dioxoethyl)-6-ethoxycarbonyl-2-ethyl-1-benzyl-1H-indol-4-yl]oxy]acetic acid,

(KK) [[3-(2-Amino-1,2-dioxoethyl)-6-n-propoxycarbonyl-2-ethyl 1-benzyl-1H-indol-4-yl]oxy]acetic acid,

(LL) [[3-(2-Amino-1,2-dioxoethyl)-6-i-propoxycarbonyl-2-ethyl-1-benzyl-1H-indol-4-yl]oxy]acetic acid,

(MM) [[3-(2-Amino-1,2-dioxoethyl)-6-cyclopropyloxycarbonyl-2-ethyl-1-benzyl-1H-indol-4-yl]oxy]acetic acid,

(NN) mixtures of (HH) through (MM) in any combination.

Most preferred in the practice of the method or composition of the invention include the following:

(I) [[3-(2-Amino-1,2-dioxoethyl)-2-ethyl-1-(phenylmethyl)-1H-indol-4-yl]oxy]acetic acid,

(I′) [[3-(2-Amino-1,2-dioxoethyl)-2-ethyl-1-(phenylmethyl)-1H-indol-4-yl]oxy]acetic acid methyl ester.

Similarly, Most preferred in the practice of the method or composition of the invention include the following:

(A) [[3-(2-Amino-1,2-dioxoethyl)-2-methyl-1-(phenylmethyl)-1H-indol-4-yl]oxy]acetic acid,

(D) [[3-(2-Amino-1,2-dioxoethyl)-1-([1,1′-biphenyl]-3-ylmethyl)-2-methyl-1H-indol-4-yl]oxy]acetic acid,

(H) [[3-(2-Amino-1,2-dioxoethyl)-2-methyl-1-[(1-naphthalenyl)methyl]-1H-indol-4-yl]oxy]acetic acid,

(J) [[3-(2-Amino-1,2-dioxoethyl)-1-[(3-chlorophenyl)methyl]-2-ethyl-1H-indol-4-yl]oxy]acetic acid,

(K) [[3-(2-Amino-1,2-dioxoethyl)-1-([1,1′-biphenyl]-2-ylmethyl)-2-ethyl-1H-indol-4-yl]oxy]acetic acid.

Similarly, Most preferred in the practice of the method or composition of the invention include the following:

(AA) (8-(Carboxymethyloxy)-2-ethyl-3-(o-phenylbenzyl)indolizin-1-yl)glyoxylamide,

(AA′) (8-Carbomethoxymethyloxy)-2-ethyl-3-(o-phenylbenzyl)indolizin-1-yl)glyoxylamide.

Similarly, Most preferred in the practice of the method or composition of the invention include the following:

(HH) [[3-(2-Amino-1,2-dioxoethyl)-6-carboxyl-2-ethyl-1-benzyl-1H-indol-4-yl]oxy]acetic acid,

(II) [[3-(2-Amino-1,2-dioxoethyl)-6-methoxycarbonyl-2-ethyl-1-benzyl-1H-indol-4-yl]oxy]acetic acid.

Most preferred in the practice of the method or composition of the invention are 1H-indole-3-glyoxylamides selected from the formula:

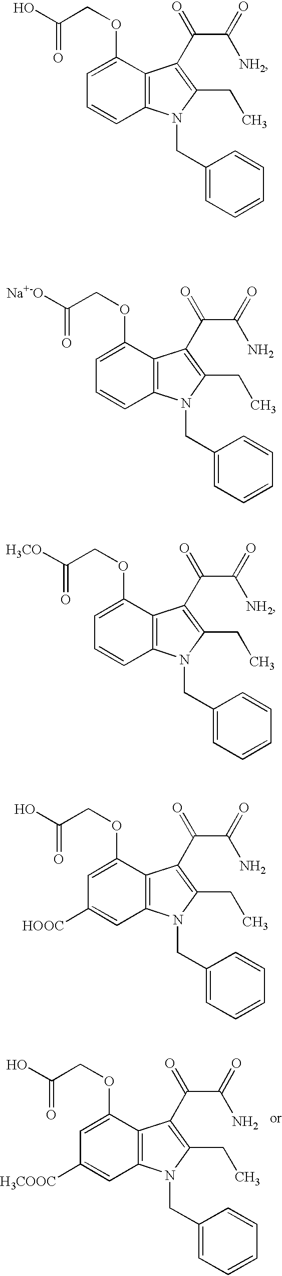

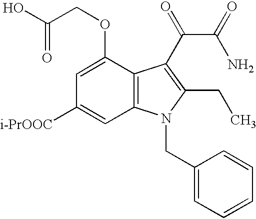

or indolizine-1-glyoxylamides selected from the formula:

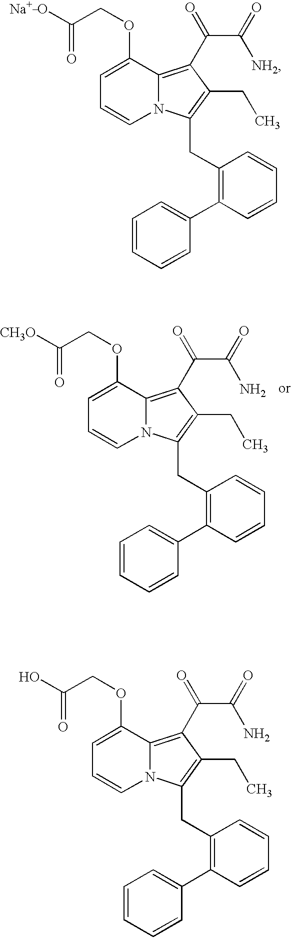

The salts of the above 1H-indole-3-glyoxylamide compounds represented by formula (II) and named compounds (A) through (P), (HH) through (NN) and of indolizine-1-glyoxylamide compounds represented by the formula (III) and named compounds (Q) through (GG) are particularly useful in the method of the invention. In those instances where the 1H-indole-3-glyoxylamide compounds and indolizine-1-glyoxylamide compounds possess acidic or basic functional groups various salts may be formed which are more water soluble and physiologically suitable than the parent compounds. Representative pharmaceutically acceptable salts, include but are not limited to, the alkali and alkaline earth salts such as lithium, sodium, potassium, calcium, magnesium, aluminum and the like. Salts are conveniently prepared from the free acid by treating the acid in solution with a base or by exposing the acid to an ion exchange resin.

Included within the definition of pharmaceutically acceptable salts are the relatively non-toxic, inorganic and organic base addition salts of the 1H-indole-3-glyoxylamide compounds and indolizine-1-glyoxylamide compounds used in the method or composition of the present invention, for example, ammonium, quaternary ammonium, and amine cations, derived from nitrogenous bases of sufficient basicity to form salts with the compounds of this invention (see, for example, S. M. Berge, et al., “Pharmaceutical Salts,” J. Phar. Sci., 66: 1-19 (1977)). Moreover, basic group(s) present in the 1H-indole-3-glyoxylamide compound may be reacted with suitable organic or inorganic acids to form salts such as acetate, benzenesulfonate, benzoate, bicarbonate, bisulfate, bitartrate, borate, bromide, camsylate, carbonate, chloride, clavulanate, citrate, chloride, edetate, edisylate, estolate, esylate, fluoride, fumarate, gluceptate, gluconate, glutamate, glycolylarsanilate, hexylresorcinate, bromide, chloride, hydroxynaphthoate, iodide, isothionate, lactate, lactobionate, laurate, malate, malate, mandelate, mesylate, methylbromide, methylnitrate, methylsulfate, mucate, napsylate, nitrate, oleate, oxalate, palmitate, pantothenate, phosphate, polygalacturonate, salicylate, stearate, subacetate, succinate, tannate, tartrate, tosylate, trifluoroacetate, trifluoromethane sulfonate, and valerate.

Certain 1H-indole-3-glyoxylamide compounds and indolizine-1-glyoxylamide compounds may possess one or more chiral centers and may thus exist in optically active forms. Likewise, R- and S-isomers and mixtures thereof, including racemic mixtures as well as mixtures of cis- and trans-isomers, are contemplated for use by the method or composition of this invention.

Prodrugs are derivatives of the 1H-indole-3-glyoxylamide compounds or indolizine-1-glyoxylamide compounds which have chemically or metabolically cleavable groups and become by solvolysis or under physiological conditions the compounds of the invention which are pharmaceutically active in vivo. Derivatives of the 1H-indole-3-glyoxylamide compounds and indolizine-1-glyoxylamide compounds have activity in both their acid and base derivative forms, but the acid derivative form often offers advantages of solubility, tissue compatibility, or delayed release in a mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acidic compound with a suitable alcohol, or amides prepared by reaction of the parent acid compound with a suitable amine. Simple aliphatic or aromatic esters (e.g., methyl or ethyl esters) derived from acidic groups (e.g., carboxyl) pendent on the compounds of this invention are preferred prodrugs. In some cases it is desirable to prepare double ester type prodrugs such as (acyloxy) alkyl esters or ((alkoxycarbonyl)oxy)alkyl esters.

The method of the invention can be practiced using pharmaceutical formulations containing compounds of the invention administered through the skin by an appliance such as a transdermal patch, as described in U.S. Pat. Nos. 5,296,222 and 5,271,940, the disclosures of which are incorporated herein by reference. Lipophilic prodrug derivatives of the compounds for formula II are particularly well suited for transdermal absorption administration and delivery systems.

The synthesis of the 1H-indole-3-glyoxylamide compounds may be accomplished as described European Patent Application No. 95302166.4, Publication No. 0 675 110 (publ., Oct. 4, 1995). Further, the synthesis of the indole dicarboxylic acid derivatives may be accomplished as described Japanese Patent Application No. 35984/1997. The synthesis of the indolizine compounds may be accomplished as described WO 9603383 (Publ., Feb. 8, 1996). Such synthetic methods also include well-known methods as recorded in the chemical literature and the procedure illustrated in the following preparative reaction scheme.

The following abbreviations are used throughout the synthesis Schemes and Examples.

| | |
|---|---|
| Et | ethyl |
| Pr | propyl |
| t-Bu | t-butyl |
| Bn | benzyl |
| LAH | lithium aluminum hydride |
| THF | tetrahydrofuran |
| DMF | dimethylformamide |
| Hex | hexyl |

Preparative Reaction Scheme 1

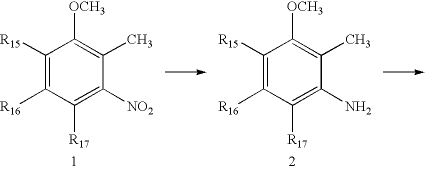

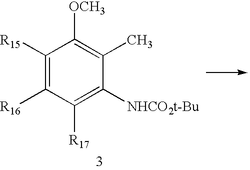

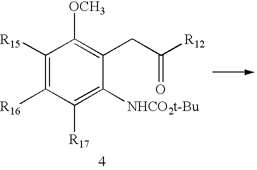

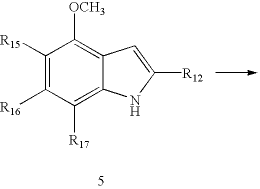

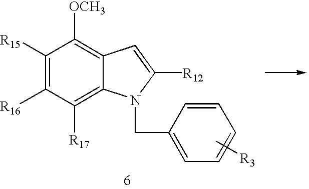

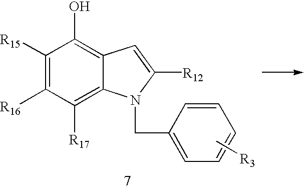

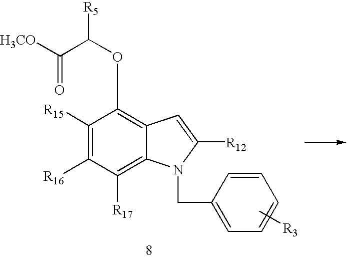

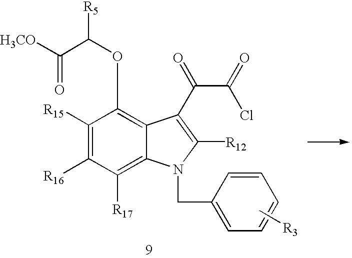

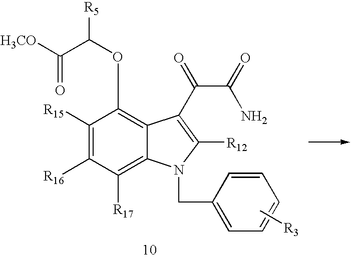

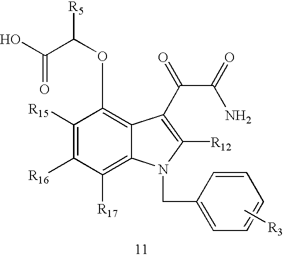

wherein

R₁₂, R₁₅, R₁₆ and R₁₇ are as defined above. R₃ Is C₁-C₅ alkyl, aryl, C₁-C₆ alkoxy, halo, aryloxy, aralkyloxy, nitro, hydroxy, amino, methylamino or dimethylamino. R₅ is hydrogen, C₁-C₁₀ alkyl, aryl, C₁-C₁₀ alkaryl, C₁-C₁₀ aralkyl or halo.

Explanation of Preparative Reaction Scheme 1:

To obtain the glyoxylamides substituted in the 4-position with an acidic function through an oxygen atom, the reactions outlined in scheme 1 are used (for conversions 1 through 5, see ref. Robin D. Clark, Joseph M. Muchowski, Lawrence E. Fisher, Lee A. Flippin, David B. Repke, Michel Souchet, Synthesis, 1991, 871-878, the disclosures of which are incorporated herein by reference). The ortho-nitrotoluene, 1, is readily reduced to the 2-methylaniline, 2, using Pd/C as catalyst. The reduction can be carried out in ethanol or tetrahydrofuran (THF) or a combination of both, using a low pressure of hydrogen. The aniline, 2, on heating with di-tert-butyl dicarbonate in THF at reflux temperature is converted to the N-tert-butylcarbonyl derivative, 3, in good yield. The dilithium salt of the dianion of 3 is generated at −40 to −20° C. in THF using sec-butyl lithium and reacted with the appropriately substituted N-methoxy-N-methylalkanamide. This product, 4, may be purified by crystallization from hexane, or reacted directly with trifluoroacetic acid in methylene chloride to give the 1,3-unsubstituted indole 5. The 1,3-unsubstituted indole 5 is reacted with sodium hydride in dimethylformamide at room temperature (20-25° C.) for 0.5-1.0 hour. The resulting sodium salt of 5 is treated with an equivalent of arylmethyl halide and the mixture stirred at a temperature range of 0-100° C., usually at ambient room temperature, for a period of 4 to 36 hours to give the 1-arylmethylindole, 6. This indole, 6, is O-demethylated by stirring with boron tribromide in methylene chloride for approximately 5 hours (see ref. Tsung-Ying Shem and Charles A Winter, Adv. Drug Res., 1977, 12, 176, the disclosure of which is incorporated herein by reference). The 4-hydroxyindole, 7, is alkylated with an alpha bromoalkanoic acid ester in dimethylformamide (DMF) using sodium hydride as a base, with reactions conditions similar to that described for the conversion of 5 to 6. The a-[(indol-4-yl)oxy]alkanoic acid ester, 8, is reacted with oxalyl chloride in methylene chloride to give 9, which is not purified but reacted directly with ammonia to give the glyoxlamide 10. This product is hydrolyzed using 1N sodium hydroxide in MeOH. The final glyoxylamide, 11, is isolated either as the free carboxylic acid or as its sodium salt or in both forms.

Preparative Reaction Scheme 2-1

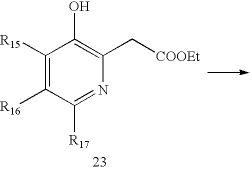

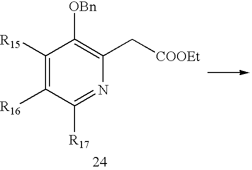

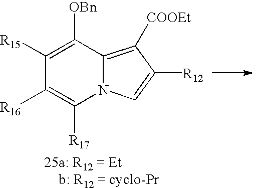

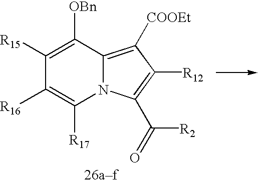

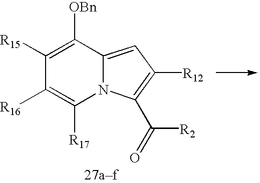

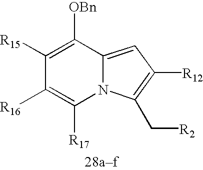

| 26-28 | R₁₂ | R₂ |
|---|---|---|
| a: | Et | Ph |
| b: | Et | o-Ph—Ph |
| c: | Et | m-Cl—Ph |
| d: | Et | m-CF₃—Ph |
| e: | Et | 1-Naphthyl |
| f: | cyclo-Pr | o-Ph—Ph |

wherein

R₁₂, R₁₅, R₁₆ and R₁₇ are defined above. R₂ is C₆-C₂₀ alkyl, C₆-C₂₀ alkenyl, C₆-C₂₀ alkynyl or carbocyclic radical.

Explanation of Preparative Reaction Scheme 2-1:

Compound 23 (N. Desideri F. Mama, M. L. Stein, G. Bile, W. Filippeelli, and E. Marmo, Eur. J. Med. Chem. Chim. Ther., 18, 295, (1983)) is O-alkylated using sodium hydride and benzyl chloride to give 24. N-alkylation of 24 by 1-bromo-2-butanone or chloromethylcyclopropyl ketone and subsequent base catalyzed cyclization gives 25 which is acylated by aroyl halide to give 26. Hydrolysis of the ester function of 26 followed by acidification forms an acid which is thermally decarboxylated to give 27. Reduction of the ketone function of 27 by LAH yields indolizines 28.

Preparative Reaction Scheme 2-2

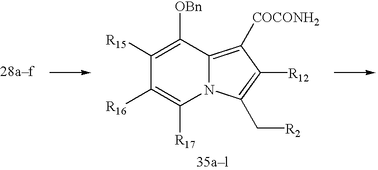

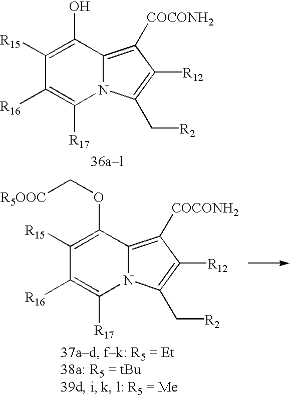

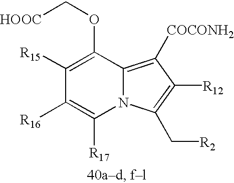

| 35-40 | R₃ | R₄ | R₁₂ | R₂ |
|---|---|---|---|---|
| a: | H | H | Et | Ph |
| d: | H | H | Et | o-Ph—Ph |
| g: | H | H | Me | Ph |
| h: | H | H | Et | m-Cl—Ph |
| i: | H | H | Et | m-CF₃—Ph |
| j: | H | H | Et | 1-Naphthyl |
| k: | H | H | cyclo-Pr | o-Ph—Ph |
| l: | H | H | Me | cyclo-Hex |

wherein

R₂, R₁₂, R₁₅, R₁₆ and R₁₇ are as defined above. R₅ is hydrogen or C₁-C₆ alkyl.

Explanation of Preparative Reaction Scheme 2-2:

Sequential treatment of 28 with oxalyl chloride and ammonium hydroxide forms 35 which is debenzylated by hydrogen in the presence of Pd/C to give 36. Indolizines 36 are O-alkylated using sodium hydride and bromoacetic acid esters to form 37, 38, or 39 which are converted to indolizines 40 by hydrolysis with aqueous base followed by acidification.

Pharmaceutical Formulations

Suitable pharmaceutical formulation of the 1H-indole-3-glyoxylamide compounds may be made as described European Patent Application No. 95302166.4, Publication No. 0 675 110 (publ., Oct. 4, 1995). Suitable pharmaceutical formulation of the indolizine-1-glyoxylamide compounds may be made as described WO 9603383 (publ., Feb. 8, 1996). Formulations may be obtained by conventional procedures well known in the pharmaceutical art.

The 1H-indole-3-glyoxylamide compound or indolizine-1-glyoxylamide compound is generally administered as an appropriate pharmaceutical composition which comprises a therapeutically effective amount of 1H-indole-3-glyoxylamide compound or indolizine-1-glyoxylamide is together with a pharmaceutically acceptable diluent or carrier, the composition being adapted for the particular route of administration chosen. By “pharmaceutically acceptable” it is meant the carrier, diluent or excipient must be compatible with the 1H-indole-3-glyoxylamide compound or indolizine-1-glyoxylamide compound in the formulation and not deleterious to the subject being treated.

Preferably the pharmaceutical formulation is in unit dosage form.

The unit dosage form can be a capsule or tablet itself, or the appropriate number of any of these. The quantity of active ingredient in a unit dose of composition may be varied or adjusted from about 0.1 to about 1000 milligrams or more according to the particular treatment involved.

The compound can be administered by a variety of routes including oral, aerosol, rectal, transdermal, subcutaneous, intravenous, intramuscular, and intranasal.

For the pharmaceutical formulations any suitable carrier known in the art can be used. In such a formulation, the carrier may be a solid, liquid, or mixture of a solid and a liquid. A solid carrier can be one or more substances which may also act as flavoring agents, lubricants, solubilisers, suspending agents, binders, tablet disintegrating agents and encapsulating material.

Tablets for oral administration may contain suitable excipients such as calcium carbonate, sodium carbonate, lactose, calcium phosphate, together with disintegrating agents, such as maize, starch, or alginic acid, and/or binding agents, for example, gelatin or acacia, and lubricating agents such as magnesium stearate, stearic acid, or talc. In tablets the 1H-indole-3-glyoxylamide compound or indolizine-1-glyoxylamide compound is mixed with a carrier having the necessary binding properties in suitable proportions and compacted in the shape and size desired. The powders and tablets preferably contain from about 1 to about 99 weight percent of the 1H-indole-3-glyoxylamide compound or indolizine-1-glyoxylamide compound.

Sterile liquid form formulations include suspensions, emulsions, syrups and elixirs. The active ingredient can be dissolved or suspended in a pharmaceutically acceptable carrier, such as sterile water, sterile organic solvent or a mixture of both.

### EXAMPLES

The following Example 1 illustrates the preparation of [[3-(2-Amino-1,2-dioxoethyl)-2-ethyl-1-(phenylmethyl)-1H-indol-4-yl]oxy]acetic acid, a 1H-indole-3-glyoxylamide compound useful in the practice of the method of the invention:

### Example 1

### Preparation of [[3-(2-Amino-1,2-dioxoethyl)-2-ethyl-1-(phenylmethyl)-1H-indol-4-yl]oxy]acetic acid, a Compound Represented by the Formula:

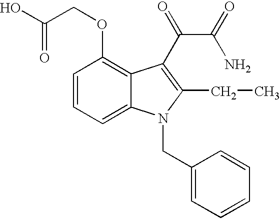

Part A. Preparation of 2-Ethyl-4-methoxy-1H-indole

A solution of 140 mL (0.18 mol) of 1.3M sec-butyl lithium in cyclohexane was added slowly to N-tert-butoxycarbonyl-3-methoxy-2-methylaniline (21.3 g, 0.09 mol) in 250 mL of THF keeping the temperature below −40° C. with a dry ice-ethanol bath. The bath was removed and the temperature allowed to rise to 0° C. and then the bath replaced. After the temperature had cooled to −60° C., 18.5 g (0.18 mol) of N-methoxy-N-methylpropanamide in an equal volume of THF was added dropwise. The reaction mixture was stirred 5 minutes, the cooling bath removed and stirred an additional 18 hours. It was then poured into a mixture of 300 mL of ether and 400 mL of 0.5N HCl. The organic layer was separated, washed with water, brine, dried over MgSO₄, and concentrated at reduced pressure to give 25.5 g of a crude of 1-[2-(tert-butoxycarbonylamino)-6-methoxyphenyl]-2-butanone. This material was dissolved in 250 mL of methylene chloride and 50 mL of trifluoroacetic acid and stirred for a total of 17 hours. The mixture was concentrated at reduced pressure and ethyl acetate and water added to the remaining oil. The ethyl acetate was separated, washed with brine, dried (MgSO₄) and concentrated. The residue was chromatographed three times on silica eluting with 20% EtOAc/hexane to give 13.9 g of 2-ethyl-4-methoxy-1H-indole.

Analyses for C₁₁H₁₃NO:

| | | | |
|---|---|---|---|
| Calculated: | C, 75.40; | H, 7.48; | N, 7.99 |
| Found: | C, 74.41; | H, 7.64; | N, 7.97. |

Part B. Preparation of 2-Ethyl-4-methoxy-1-(phenylmethyl)-1H-indole

2-Ethyl-4-methoxy-1H-indole (4.2 g, 24 mmol) was dissolved in 30 mL of DMF and 960 mg (24 mmol) of 60% NaH/mineral oil was added. After 1.5 hours, 2.9 mL (24 mmol) of benzyl bromide was added. After 4 hours, the mixture was diluted with water and extracted twice with ethyl acetate. The combined ethyl acetate was washed with brine, dried (MgSO₄) and concentrated at reduced pressure. The residue was chromatographed on silica gel and eluted with 20% EtOAc/hexane to give 3.1 g (49% yield) of 2-ethyl-4-methoxy-1-(phenylmethyl)-1H-indole.

Part C. Preparation of 2-Ethyl-4-hydroxy-1-(phenylmethyl)-1H-indole

By the method used in Example 1, Part D, in EP Publication No. 0 675 110, 3.1 g (11.7 mmol) of 2-ethyl-4-methoxy-1-(phenylmethyl)-1H-indole was O-demethylated by treating it with 48.6 mL of 1M BBr₃/CH₂Cl₂ to give a material that was chromatographed on silica gel (eluted with 20% EtOAc/hexane) to give 1.58 g (54% yield) of 2-ethyl-4-hydroxy-1-(phenylmethyl)-1H-indole, mp, 86-90° C.

Analyses for C₁₇H₁₇NO:

| | | | |
|---|---|---|---|
| Calculated: | C, 81.24; | H, 6.82; | N, 5.57 |
| Found: | C, 81.08; | H, 6.92; | N, 5.41. |

Part D. Preparation of 2-[[2-Ethyl-1-(phenylmethyl)-1H-indol-4-yl]oxy]acetic acid methyl ester

Using the procedure described in Example 1, Part E, in EP Publication No. 0 675 110, 2-ethyl-4-hydroxy-1-(phenylmethyl)-1H-indole (1.56 g, 6.2 mmol) was treated with 248 mg (6.2 mmol) of 60% NaH/mineral oil and then 0.6 mL (6.2 mmol ) of methyl bromoacetate. The product was purified by chromatography over silica gel eluting with 20% EtOAc/hexane, to give 1.37 g (69% yield) of [[2-ethyl-1-(phenylmethyl)-1H-indol-4-yl]oxyl]acetic acid methyl ester, ; mp 89-92° C.

Analyses for C₂₀H₂₁NO₃:

| | | | |
|---|---|---|---|
| Calculated: | C, 74.28; | H, 6.55; | N, 4.33 |
| Found: | C, 74.03; | H, 6.49; | N, 4.60. |

Part E. Preparation of [[3-(2-Amino-1,2-dioxoethyl)-2-ethyl-1-(phenylmethyl)-1H-indol-4-yl]oxy]acetic acid methyl ester

Using the procedure in Example F, in EP Publication No. 0 675 110, 1.36 g (4.2 mmol) of [[2-ethyl-1-(phenylmethyl)-1H-indol-4-yl]oxy]acetic acid methyl ester was reacted first with 0.4 mL (4.2 mmol) of oxalyl chloride and then excess ammonia to give a white solid. This was stirred with ethyl acetate and the insoluble material separated and dried to give 1.37 g of a mixture of [[3-(2-amino-1,2-dioxoethyl)-2-ethyl-1-(phenylmethyl)-1H-indol-4-yl]oxy]acetic acid methyl ester and ammonium chloride. This mixture melted at 172-187° C.

Part F. Preparation of [[3-(2-Amino-1,2-dioxoethyl)-2-ethyl-1-(phenylmethyl)-1H-indol-4-yl]oxy]acetic acid

A mixture of 788 mg (2 mmol) of [3-(2-Amino-1,2-dioxoethyl)-2-ethyl-1-(phenylmethyl)-1H-indol-4-yl]oxy]acetic acid methyl ester, 10 mL of 1n NaOH and 30 mL of MeOH was heated to maintain reflux for 0.5 hour, stirred at room temperature for 0.5 hour and concentrated at reduced pressure. The residue was taken up in ethyl acetate and water, the aqueous layer separated and made acidic to pH 2-3 with 1N HCl. The precipitate was filtered and washed with ethyl acetate to give 559 mg (74% yield) of [[3-(2-Amino-1,2-dioxoethyl)-2-ethyl-1-(phenylmethyl)-1H-indol-4-yl]oxy]acetic acid, mp 230-234° C.

Analyses for C₂₁H₂₀N₂O₅:

| | | | |
|---|---|---|---|
| Calculated: | C, 65.96; | H, 5.80; | N, 7.33 |
| Found: | C, 66.95; | H, 5.55; | N, 6.99. |

The following Example 2 illustrates the preparation of (8-(Carboxymethyloxy)-2-ethyl-3-(o-phenylbenzyl)indolizin-1-yl)glyoxylamide, a indolizine-1-glyoxylamide compound useful in the practice of the method of the invention:

### Example 2

### Preparation of (8-(Carboxymethyloxy)-2-ethyl-3-(o-phenylbenzyl)indolizin-1-yl)glyoxylamide, a Compound Represented by the Formula

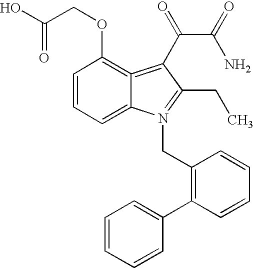

Part A: Preparation of Ethyl 3-benzyloxy-2-pyridineacetate 24

60% Sodium hydride (2.69 g, 66.2 m mol) was added in small portions to a solution of ethyl 3-hydroxy-2-pyridineacetate (23, 12.0 g, 66.2 mmol) (N. Desideri, F. Manna, M. L. Stein, G. Bile, W. Filippeelli, and E. Marmo. Eur. J. Med. Chem. Chim. Ther., 18, 295 (1983)) in dimethylformamide (220 ml) at 0° C. The mixture was stirred at 0° C. for 50 min. Benzyl chloride (8.4 ml, 72.8 mmol) was added dropwise to the mixture, which was stirred overnight. Ethyl acetate was added. The mixture was washed with 5% aqueous sodium hydrogencarbonate and water and dried over Na₂SO₁. After removing the solvent at reduced pressure, the residue was chromatographed on silica gel eluting with AcOEt:toluene (1:19 to 1:1) to give 16.17 g (90.0% yield) of the titled compound as an oil.

IR νₘₐₓ (film) 1736, 1446, 1278 cm⁻¹. ¹H NMR (CDCl₃) δ1.21 (3H, t, J=7.2 Hz), 3.93 (2H, s), 4.14 (2H, q, J=7.2 Hz), 5.10 (2H, s), 7.13-7.22 (2H, m), 7.32-7.43 (5H, m), 8.16 (1H, dd, J=4.0, 3.0 Hz). Analyses: Calc'd for C₁₆H₁₇NO₃: C, 70.83; H, 6.32; N, 5.16. Found: C, 70.65; H, 6.37; N, 5.20.

Part B: Preparation of Ethyl (8-benzyloxy-2-ethylindolizin-1-yl)carboxylate 25a

A mixture of pyridine derivative (24, 15.15 g, 55.8 mmol) sodium hydrogencarbonate (23.45 g, 279 mmol) and 1-bromo-2-butanone (11.4 ml, 113 mmol) in methylethylketone (250 ml) was heated under reflux for 24 hours, washed with water and dried over Na₂SO₁. After removing the solvent at reduced pressure, the residue was chromatographed on silica gel eluting with AcOEt:hexane (1:19 to 1:9) to give 16.66 g, (92.0% yield) of the titled compound as an oil.

IR νₘₐₓ (film) 1690, 1227, 1092 cm⁻¹. ¹H NMR (CDCl₃) δ1.15 (3H, t, J=7.2 Hz), 1.26 (3H, t, J=7.5 Hz), 2.82 (2H, q, J=7.5 Hz), 4.11 (2H, q, J=7.2 Hz), 5.16 (2H, s), 6.22 (1H, d, J=7.6 Hz), 6.44 (1H, t, J=7.1 Hz), 7.07 (1H, s), 7.27-7.57 (6H, m). Analyses: Calc'd for C₂₀H₂₁NO₃ 0.1H₂O: C, 73.87; H, 6.57; N, 4.31. Found: C, 73.75; H, 6.66; N, 4.30.

Part C: Preparation of Ethyl (8-benzyloxy-2-ethyl-3-(o-phenylbenzoyl)indolizin-1-yl)carboxylate 26b

A mixture of the indolizine (25, l eq), o-phenyl benzoyl chloride (2.0 eq) and triethylamine (5.0 eq) was heated at 90° C. (bath temp.) for 2-8 hours. Ethyl acetate was added. The mixture was washed with dilute hydrochloric acid and water and dried over Na₂SO₁. After removing the solvent at reduced pressure, the residue was chromatographed on silica gel eluting with AcOEt:hexane (1:2) and recrystallized.

mp, 110-112° C. (ether-hexane). 46.0% Yield.

Part D: Preparation of 8-Benzyloxy-2-ethyl-3-(o-phenylbenzoyl)indolizine 27b

To a solution of the ester (26, 1.0 mmol) in dimethylsulfoxide (10 ml), 50% aqueous potassium hydroxide (3 ml) was added. The mixture was heated at 140° C. for 2-24 hours. After cooling, the mixture was acidified with dilute hydrochloric acid and extracted with ethyl acetate. The extracts were washed with water dried over Na₂SO₁. After removing the solvent under reduced pressure, the residue was purified by recrystallization to give the carboxylic acid.

The acid in toluene was heated under reflux for 1 hour and the solvent was removed by distillation at reduced pressure. The residue was purified by recrystallization to give 27.

Quantitative yield. IR νₘₐₓ (nujol) 1735, 1597, 742 cm⁻¹.

Part E: Preparation of 8-Benzyloxy-2-ethyl-3-(o-phenylbenzyl)indolizine 28b

Compound 27 was treated by the procedure described for the preparation of 4, WO 9603383.

Quantitative yield. IR νₘₐₓ (CHCl₃) 1525, 1259 cm⁻¹.

Part F: Preparation of (8-Benzyloxy-2-ethyl-3-(o-phenylbenzyl)indolizin-1-yl)glyoxylamide 35d

These compounds were prepared according to the procedure described for the synthesis of compound 8 from compound 4, WO 9603383.

Mp, 183-185° C. (ether-hexane). 79.0% Yield.

Part G: Preparation of (2-Ethyl-8-hydroxy-3-(o-phenylbenzyl)indolizin-1-yl)glyoxylamide 36d

These compounds were prepared according to the procedure described for the synthesis of compound 20 from 19, WO 9603383.

Mp, 195-196° C. (dec.) (ether-hexane). 95.0% Yield.

Part H: Preparation of (8-(Carbomethoxymethyloxy)-2-ethyl-3-(o-phenylbenzyl)indolizin-1-yl)glyoxylamide 39d

These compounds were prepared according to the procedure described for the synthesis of compound 21 from 20, WO 9603383.

Mp, 73-75° C. (dec.) (ether-hexane). 84% Yield.

Part I: Preparation of (8-(Carboxymethyloxy)-2-ethyl-3-(o-phenylbenzyl)indolizin-1-yl)glyoxylamide 40d

1N-Aqueous potassium hydroxide (4 ml) was added to a solution of the ester (37-39, 2 mmol) in methanol (21 ml). The solution was stirred at room temperature for 40 min, washed with ether, acidified with 2N—HCl and extracted with ethyl acetate. The extracts were washed with water and dried over Na₂SO₄. After removing the solvent at reduced pressure, the residue was recrystallized.

Mp, 209-212° C. (dec.) (ether-hexane). 93% Yield. IR νₘₐₓ (nujol) 3316, 1704, 1601, 1493 cm⁻¹. ¹H NMR (d₆-DMSO) δ1.01 (3H, t, J=7.5 Hz), 2.67 (2H, q, J=7.5 Hz), 4.18 (2H, s), 4.71 (2H, s), 6.41 (1H, d, J=7.8 Hz), 6.57-6.59 (2H, m), 7.14-7.57 (10H, m), 7.34 (1H, s), 13.09 (1H, br.s).

Analyses: Calc'd for C₂₇H₂₄N₂O₅ 0.3H₂O: C, 70.21; H, 5.37; N, 6.06.

Found: C, 70.17; H, 5.35; N, 5.98.

The disorders associated with apoptosis treatment utility of the method of the invention will now be illustrated by the following Example 3 and 4:

### Example 3

This example illustrates the action of (8-(Carboxymethyloxy)-2-ethyl-3-(o-phenylbenzyl)indolizin-1-yl)glyoxylamide (the compound prepared in Example 2, hereinafter called “Ex-2”) for neuronal death induced by human PLA₂-II (hPLA₂-II)

(1) Primary Culture of Neuron

According to a method disclosed in Neurosci. Lett. 203, 175-178 Ueda K. et al, Neuronal cultures were prepared from cerebral cortex of 19-day-old Sprague-Dawley rat embryos. The cerebral cortices were dissociated in isotonic buffer (137 mM NaCl, 5.4 mM KCl, 0.17 mM Na2HPO4, 0.22 mM KH2PO4, 5.5 mM Glucose, 59 mM Sucrose; volume of each buffer is 25 ml) with 4 mg/ml trypsin and 0.4 mg/ml deoxyribonuclease I. Cells were plated at a density of 2.5×10⁵ cells/cm² on poly-L-lysine coated dishes in conditioning medium, Leibovitz's L-15 medium supplemented with 5% fetal calf serum and 5% horse serum. Culture medium was exchanged for conditioning medium containing 0.1 mM arabinosylcytosine C on day 1 after plating. Cultured neurons were used for the experiments on day 2 of culture.

(2) Ultrastructural Changes in Cortical Neurons After hPLA₂-II Treatment

Cells were treated with hPLA₂-II for 48 h, and fixed in situ in 1-5% (especially 2.5%) glutaraldehyde in PBS for 2 h at 4° C. and post-fixed in 0.5-2% (especially 1% osmium tetraoxide). To increase contrast, cells were double-fixed in saturated thiocarbohydrazide-osmium. Afterwards, samples were dehydrated using a graded series of ethanol from 50-100%. Tissue culture dishes were embedded in Araldite or Epoxy resin (especially Epon 812), cured in vacuo for 48 h at 60-70° C. and sectioned. Afterwards, the culture were double-dyeing with uranium acetate and lead citrate, it was identified the formation by electron microscope. The result was shown in FIG. 1. FIG. 1A shows the ultrastructure of untreated neurons, while FIG. 1B shows the ultrastructure of hPLA₂-II treated neurons.

Result: D We confirmed that untreated neurons had bright and round soma, and extended neurites. On the other hand, hPLA₂-II-treated neurons had shrank cell bodies and lost their neurites. In comparison with untreated cells (FIG. 1A), condensation and fragmentation of nuclear chromatin, lost of intracellular organelle other than mitochondria and blebbing of plasma membranes were observed in hPLA₂-II-treated cells (FIG. 1B).

(3) Analysis of Neuronal Survival

For assessment of neurotoxicity of hPLA₂-II according to a method disclosed in Neurosci. Lett. 203, 175-178 Ueda K. et al, the 3-(4,5-dimethylthiazol-2-yl)-2,5-diphenyl tetrazolium bromide dye (MTT) reduction assay reflecting mitochondrial activity was employed. MTT (5 mg/ml) was applied to the conditioning medium (vol/vol=1/50-1/100) and incubated at 37° C. for 1 hour. Supernatant was aspirated and cells were solved by isopropanol containing 0.04 N HCl (100-200 ml). Absorbance at 570 nm were measured with a microplate reader.

p-BPB : 100 μM hPLA₂-II were preincubated with 0.01, 0.1 or 1 mM p-bromophenacylbromide for 20 min at 37° C. After preincubation, an aliquot was removed and diluted 100-fold in the culture medium of cortical neutrons.

Vehicle and the compound prepared in Example 2: Cortical neurons were treated with vehicle or 1 μM hPLA₂-II in the presence of the compound prepared in Example 2 at the indicated concentrations on day 2 of culture. MTT reducing activity was determined 48 hr after hPLA₂-II-treatment.

The result was shown in FIG. 2.

Post-treatment with the compound prepared by Example 2: Cortical neurons were treated with vehicle or 1 μM hPLA₂-II on day 2 of culture. At the indicated time after the treatment with hPLA₂-II (1 μM), the compound prepared in Example 2 (final concentration=10 μM) was added to the culture medium. Data are expressed as mean±SEM values (n=4). *P<0.05, **P<0.01, compared with vehicle-treated conditions by ANOVA followed by Dunnett's test. The result was shown in FIG. 3.

Result: From FIG. 2, we confirmed that the compound prepared in Example 2 suppressed neuronal death in a concentration-dependent manner. From FIG. 3, we confirmed that the compound prepared in Example 2 suppressed neuronal death completely within 10 hours after hPLA₂-II treatment. Thus, posttreatment with a hPLA₂-II inhibitor, as well as co-treatment, could rescue neuron from hPLA₂-II-induced death. Example 4

This example illustrates the action of [[3-(2-Amino-1,2-dioxoethyl)-2-ethyl-1-(phenylmethyl)-1H-indol-4-yl]oxy]acetic acid (the compound prepared in Example 1, hereinafter called “Ex-1”) for neuronal death induced by human PLA₂-II (hPLA₂-II)

Analysis of Neuronal Survival

The experiment was carried out in the same method as in Example 3 (3) mentioned above other than adding 3 μM hPLA₂-II to 48 well-cultured neuron instead of 1 μM hPLA₂-II to 12 well-cultured one. The result was shown in FIG. 4.

Result: From FIG. 4, we confirmed that the compound prepared in Example 1 suppressed neuronal death completely depending on its concentration.

The following pharmaceutical formulations 1 through 8 are illustrative only and are not intended to limit the scope of the invention in any way. “Active ingredient”, refers to a compound according to formula (I) or a pharmaceutically acceptable salt, solvate, or prodrug thereof.

Formulation 1

Hard gelatin capsules are prepared using the following ingredients:

Quantity (mg/capsule)

| | | |
|---|---|---|
| Active ingredient | 250 | |
| Starch, dried | 200 | |
| Magnesium stearate | 10 | |
| Total | 460 | mg |

Formulation 2

A tablet is prepared using the ingredients below:

| | Quantity (mg/tablet) |
|---|---|
| Active ingredient | 250 |
| Cellulose, microcrystalline | 400 |
| Silicon dioxide, fumed | 10 |
| Stearic acid | 5 |
| Total | 665 |

Formulation 3

An aerosol solution is prepared containing the following components:

| | Weight |
|---|---|
| Active ingredient | 0.25 |
| Ethanol | 25.75 |
| Propellant 22 | 74.00 |
| (Chlorodifluoromethane) | |
| Total | 100.00 |

The active compound is mixed with ethanol and the mixture added to a portion of the propellant 22, cooled to −30° C. and transferred to a filling device. The required amount is then fed to a stainless steel container and diluted with the remainder of the propellant. The valve units are then fitted to the container.

Formulation 4

Tablets, each containing 60 mg of active ingredient, are made as follows:

| | |
|---|---|
| Active ingredient | 60 mg |
| Starch | 45 mg |
| Microcrystalline cellulose | 35 mg |
| Polyvinylpyrrolidone | 4 mg |
| (as 10% solution in water) | |
| Sodium carboxymethyl starch | 4.5 mg |
| Magnesium stearate | 0.5 mg |
| Talc | 1 mg |
| Total | 150 mg |

The active ingredient, starch and cellulose are passed through a No.45 mesh U.S.sieve and the mixed thoroughly. The aqueous solution containing polyvinylpyrrolidone is mixed with the resultant powder, and the mixture then is passed through a No.14 mesh U.S.sieve. The granules so produced are dried at 50° C. and passed through No.18 mesh U.S.sieve. The sodium carboxymethyl starch, magnesium stearate and talc, previously passed through a No.60 mesh U.S.sieve, are then added to the granules which, after mixing, are compressed on a tablet machine to yield tablets each weighing 150 mg.

Formulation 5

Capsules, each containing 80 mg of active ingredient, are made as follows:

| | |
|---|---|
| Active ingredient | 80 mg |
| Starch | 59 mg |
| Microcrystalline cellulose | 59 mg |
| Magnesium stearate | 2 mg |
| Total | 200 mg |

The active ingredient, cellulose, and magnesium stearate are blended, passed through a No.45 mesh U.S.sieve, and filled into hard gelatin capsules in 200 mg quantities.

Formulation 6

Suppositories, each containing 225 mg of active ingredient, are made as follows:

| | |
|---|---|
| Active ingredient | 225 mg |
| Saturated fatty acid glycerides | 2000 mg |
| Total | 2225 mg |

The active ingredient is passed through a No.60 mesh U.S.sieve and suspended in the saturated fatty acid glycerides previously melted using the minimum heat necessary. The mixture is then poured into a suppository mold of nominal 2 g capacity and allowed to cool.

Formulation 7

Suspensions, each containing 50 mg of active ingredient per 5 ml dose, are made as follows:

| | |
|---|---|
| Active ingredient | 50 mg |
| Sodium carboxymethyl cellulose | 50 mg |
| Syrup | 1.25 ml |
| Benzoic acid solution | 0.10 ml |
| Flavor | q.v. |
| Color | q.v. |
| Purified water to total | 5 ml |

The active ingredient is passed through a No.45 mesh U.S.sieve and mixed with the sodium carboxymethyl cellulose and syrup to form a smooth paste. The benzoic acid solution, flavor and color are diluted with a portion of the water and added, with stirring. Sufficient water is then added to produce the required volume.

Formulation 8

An intravenous formulation may be prepared as follows:

| | |
|---|---|
| Active ingredient | 100 mg |
| Isotonic saline | 1000 ml |

The solution of the above ingredients generally is administered intravenously to a subject at a rate of 1 ml per minute.

While the present invention has been illustrated above by certain specific embodiments, it is not intended that these specific examples should limit the scope of the invention as described in the appended claims.

### BRIEF DESCRIPTION OF DRAWINGS

FIG. 1A shows the ultrastructure of untreated neurons.

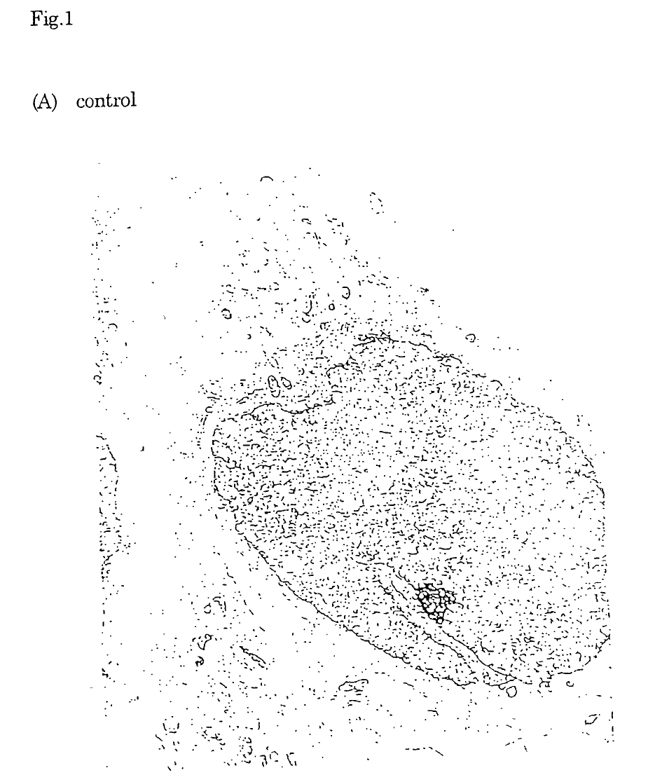

FIG. 1B shows the ultrastructure of hPLA₂-II-treated neurons.

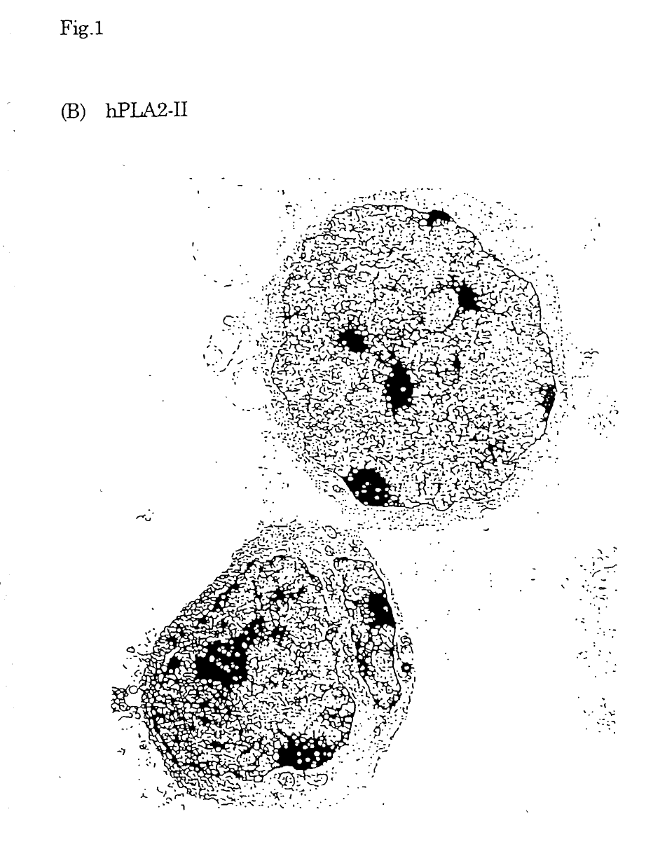

FIG. 2 shows the concentration-dependent effect of Ex-2 on hPLA₂-II-induced neuronal cell death.

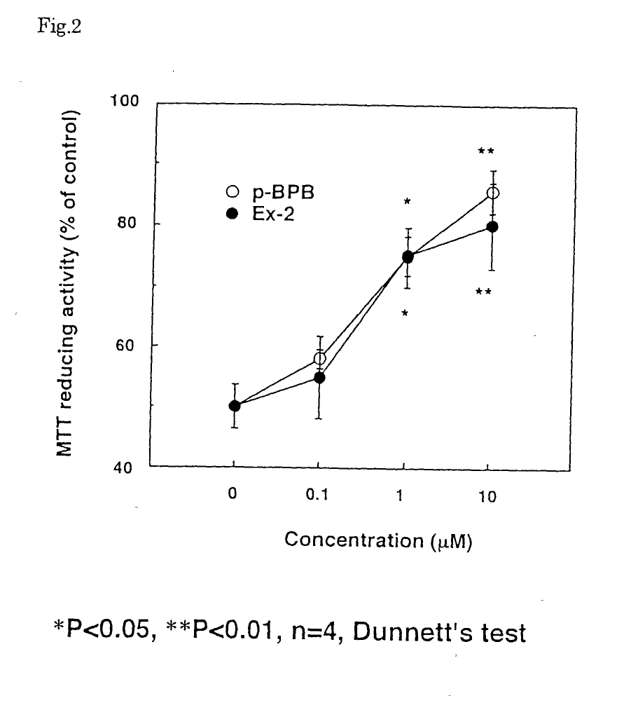

FIG. 3 shows the time-dependent effect of Ex-2 on hPLA₂-II-induced neuronal cell death.

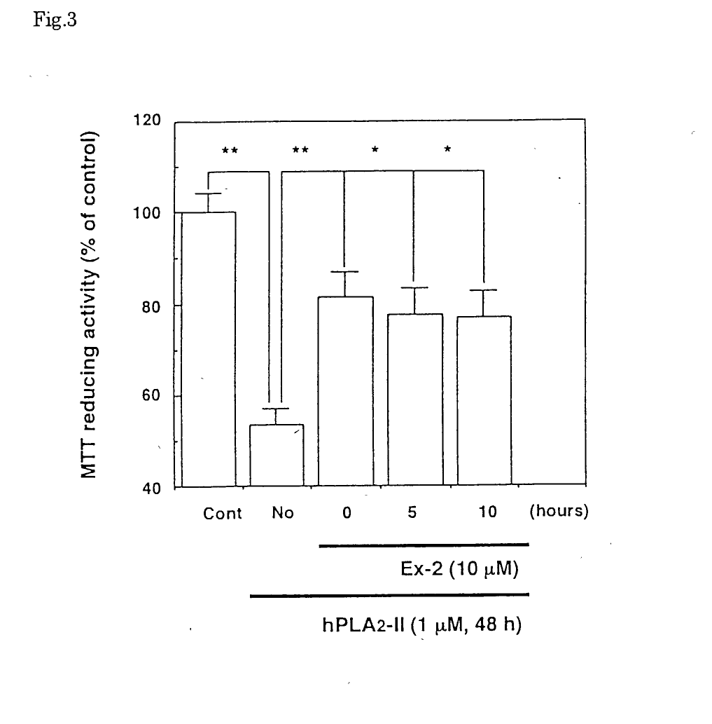

FIG. 4 shows the concentration-dependent effect of Ex-1 on hPLA₂-II-induced neuronal cell death.

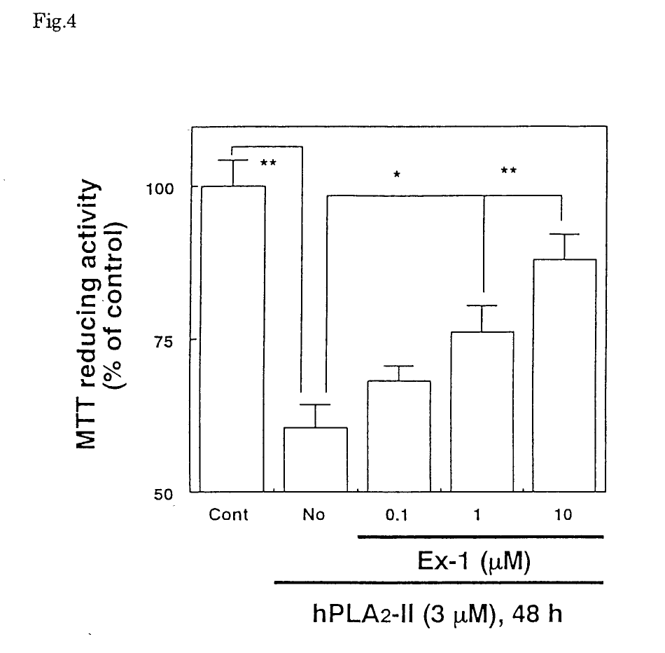

1. A method of treatment of a mammal currently afflicted with disorders associated with apoptosis or previously afflicted with disorders associated with apoptosis, said method comprising administering to said mammal a therapeutically effective amount of an N-heterocyclic glyoxylamide compound represented by the formula (I) or a pharmaceutically acceptable salt, solvate, or prodrug derivative thereof: 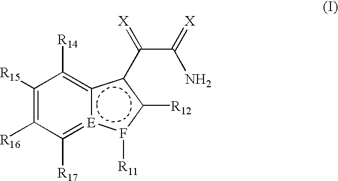
wherein;
E and F are differently C or N;
--- is presence or absence of a double bond;
each X is independently oxygen or sulfur;
R₁₁ is selected from groups (a), (b) and (c) where;
(a) is C₇-C₂₀ alkyl, C₇-C₂₀ alkenyl, C₇-C₂₀ alkynyl; or carbocyclic radical selected from the group cycloalkyl, cycloalkenyl, phenyl, naphthyl, norbornanyl, bicycloheptadienyl, tolyl, xylyl, indenyl, stilbenyl, terphenylyl, diphenylethylenyl, phenyl-cyclohexenyl, acenaphthylenyl, and anthryl, biphenyl, bibenzylyl and related bibenzylyl homologues represented by the formula (bb), 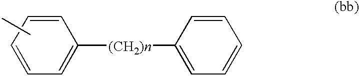
where
n is a number from 1 to 8; or
(b) is a member of (a) substituted with one or more independently selected non-interfering substituents selected from the group consisting of C₁-C₆ alkyl, C₂-C₆ alkenyl, C₂-C₆ alkynyl, C₇-C₁₂ aralkyl, C₇-C₁₂ alkaryl, C₃-C₈ cycloalkyl, C₃-C₈ cycloalkenyl, phenyl, tolyl, xylyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₁-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ—O—(C₁-C₆ alkyl), benzyloxy, phenoxy, phenylthio, —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl; where n is from 1 to 8;
(c) is the group -(L₁)-R₈₁; where, -(L₁)- is a divalent linking group having the formula; 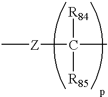
where,
R₈₄ and R₈₅ are each independently selected from hydrogen, C₁-C₁₀ alkyl, carboxyl, carbalkoxy, or halo;
p is 1 to 5,
Z is a bond, —(CH₂)—, —O—, —N(C₁-C₁₀ alkyl)-, —NH—, or —S—; and where R₈₁ is a group selected from (a) or (b);
R₁₂ is hydrogen, halo, C₁-C₃ alkyl, C₃-C₄ cycloalkyl, C₃-C₄ cycloalkenyl, —O—(C₁-C₂ alkyl), or —S—(C₁-C₂ alkyl);
R₁₄ is hydrogen or a group, -(La)-(acidic group) wherein -(Lₐ)- is represented by the formula; 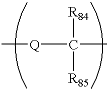
where
Q is selected from the group —(CH₂)—, —O—, —NH—, and —S—, and R₈₄ and R₈₅ are each independently selected from hydrogen, C₁-C₁₀ alkyl, aryl, C₁-C₁₀ alkaryl, C₁-C₁₀ aralkyl, and halo;
R₁₅ is hydrogen or a group, -(Lₐ′)-(acidic group) wherein -(Lₐ′)- is represented by the formula; 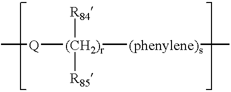
where
r is a number from 1 to 7, s is 0 or 1, and Q is selected from the group —(CH₂)—, —O—, —NH—, and —S—, and R_{84′} and R_{85′} are each independently selected from hydrogen, C₁-C₁₀ alkyl, aryl, C₁-C₁₀ alkaryl, C₁-C₁₀ aralkyl, carboxy, carbalkoxy, and halo; provided that at least one of R14 or R15 must be the group, -(La)-(acidic group) or -(La′)-(acidic group);
R₁₆ is hydrogen, carboxyl or ester thereof;
R₁₇ is selected from hydrogen, non-interfering substituents, selected from the group consisting of C₁-C₆ alkyl, C₂-C₆ alkenyl, C₂-C₆ alkynyl, C₇-C₁₂ aralkyl, C₇-C₁₂ alkaryl, C₃-C₈ cycloalkyl, C₃-C₈ cycloalkenyl, phenyl, tolyl, xylyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ—O—(C₁-C₆ alkyl), benzyloxy, phenoxy, phenylthio, —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl; where n is from 1 to 8.

2. A method of treatment of a mammal currently afflicted with disorders associated with apoptosis or previously afflicted with disorders associated with apoptosis, said method comprising administering to said mammal a therapeutically effective amount of a 1H-indole-3-glyoxylamide compound represented by the formula (II) or a pharmaceutically acceptable salt, solvate, or prodrug derivative thereof: 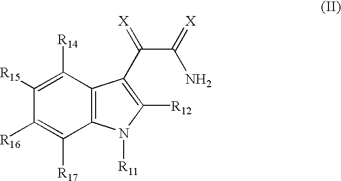
wherein
X, R₁₁, R₁₂, R₁₄, R₁₅, R₁₆ and R₁₇ are as defined above.

3. A method of treatment of a mammal currently afflicted with disorders associated with apoptosis or previously afflicted with disorders associated with apoptosis, said method comprising administering to said mammal a therapeutically effective amount of an indolizine-1-glyoxylamide compound represented by the formula (III) or a pharmaceutically acceptable salt, solvate, or prodrug derivative thereof: 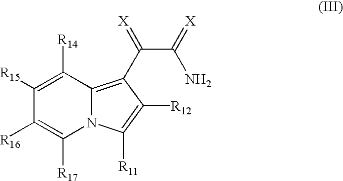
wherein
X, R₁₁, R₁₂, R₁₄, R₁₅, R₁₆ and R₁₇ are as defined above.

4. The method of claim 2 or 3 wherein for the compound of formula (II) or (III) both X are oxygen, only one of R₁₄ or R₁₅ are -(La)-(acidic group) or -(La′)-(acidic group) and the (acidic group) is carboxyl.

5. A method of treatment of a mammal currently afflicted with disorders associated with apoptosis or previously afflicted with disorders associated with apoptosis, said method comprising administering to said mammal in need of such treatment a therapeutically effective amount of an N-heterocyclic glyoxylamide compound or a pharmaceutically acceptable salt, solvate, or a prodrug derivative thereof selected from the group consisting of compounds (A) through (NN):
(A) [[3-(2-Amino-1,2-dioxoethyl)-2-methyl-1-(phenylmethyl)-1H-indol-4-yl]oxy]acetic acid,
(B) dl-2-[[3-(2-Amino-1,2-dioxoethyl)-2-methyl-1-(phenylmethyl)-1H-indol-4-yl]oxy]propanoic acid,
(C) [[3-(2-Amino-1,2-dioxoethyl)-1-([1,1′-biphenyl]-2-ylmethyl)-2-methyl-1H-indol-4-yl]oxy]acetic acid,
(D) [[3-(2-Amino-1,2-dioxoethyl)-1-([1,1′-biphenyl]-3-ylmethyl)-2-methyl-1H-indol-4-yl]oxy]acetic acid,
(E) [[3-(2-Amino-1,2-dioxoethyl)-1-([1,1′-biphenyl]-4-ylmethyl)-2-methyl-1H-indol-4-yl]oxy]acetic acid,
(F) [[3-(2-Amino-1,2-dioxoethyl)-1-[(2,6-dichlorophenyl)methyl]-2-methyl-1H-indol-4-yl]oxy]acetic acid,
(G) [[3-(2-Amino-1,2-dioxoethyl)-1-[(4-fluorophenyl)methyl]-2-methyl-1H-indol-4-yl]oxy]acetic acid,
(H) [[3-(2-Amino-1,2-dioxoethyl)-2-methyl-1-[(1-naphthalenyl)methyl]-1H-indol-4-yl]oxy]acetic acid,
(I) [[3-(2-Amino-1,2-dioxoethyl)-2-ethyl-1-(phenylmethyl)-1H-indol-4-yl]oxy]acetic acid,
(I′) [[3-(2-Amino-1,2-dioxoethyl)-2-ethyl-1-(phenylmethyl)-1H-indol-4-yl]oxy]acetic acid methyl ester
(J) [[3-(2-Amino-1,2-dioxoethyl)-1-[(3-chlorophenyl)methyl]-2-ethyl-1H-indol-4-yl]oxy]acetic acid,
(K) [[3-(2-Amino-1,2-dioxoethyl)-1-([1,1′-biphenyl]-2-ylmethyl)-2-ethyl-1H-indol-4-yl]oxy]acetic acid,
(L) [[3-(2-amino-1,2-dioxoethyl)-1-([1,1′-biphenyl]-2-ylmethyl)-2-propyl-1H-indol-4-yl]oxy]acetic acid,
(M) [[3-(2-Amino-1,2-dioxoethyl)-2-cyclopropyl-1-(phenylmethyl)-1H-indol-4-yl]oxy]acetic acid,
(N) [[3-(2-Amino-1,2-dioxoethyl)-1-([1,1′-biphenyl]-2-ylmethyl)-2-cyclopropyl-1H-indol-4-yl]oxy]acetic acid,
(O) 4-[[3-(2-Amino-1,2-dioxoethyl)-2-ethyl-1-(phenylmethyl)-1H-indol-5-yl]oxy]butanoic acid,
(P) mixtures of (A) through (O),
(Q) (8-(Carbomethoxymethyloxy)-2-ethyl-3-(o-phenylbenzyl)indolizin-1-yl)glyoxylamide,
(R) (3-Benzyl-8-(carbethoxymethyloxy)-2-ethylindolizin-1-yl)glyoxylamide,
(S) (8-(Carbethoxymethyloxy)-2-ethyl-3-(o-phenylbenzyl)indolizin-1-yl)glyoxylamide,
(T) (3-Benzyl-8-(carbethoxymethyloxy)-2-methylindolizin-1-yl)glyoxylamide,
(U) (8-(Carbethoxymethyloxy)-3-(m-chlorobenzyl)-2-ethylindolizin-1-yl)glyoxylamide,
(V) (8-Carbethoxymethyloxy-2-ethyl-3-(1-naphthylmethyl)indolizin-1-yl)glyoxylamide,
(W) (3-Benzyl-8-(t-butoxycarbonylmethyloxy)-2-ethylindolizin-1-yl)glyoxylamide,
(X) (8-(Carbmethoxymethyloxy)-2-ethyl-3-(m-trifluoromethylbenzyl)indolizin-1-yl)glyoxylamide,
(Y) (8-(Carbmethoxymethyloxy)-2-cyclopropyl-3-(o-phenylbenzyl)indolizin-1-yl)glyoxylamide,
(Z) (3-Benzyl-8-(carboxymethyloxy)-2-ethylindolizin-1-yl)glyoxylamide,
(AA) (8-(Carboxymethyloxy)-2-ethyl-3-(o-phenylbenzyl)indolizin-1-yl)glyoxylamide,
(AA′) (8-Carbomethoxymethyloxy)-2-ethyl-3-(o-phenylbenzyl)indolizin-1-yl)glyoxylamide,
(BB) (3-Benzyl-8-(carboxymethyloxy)-2-methylindolizin-1-yl)glyoxylamide,
(CC) (8-(Carboxymethyloxy)-3-(m-chlorobenzyl)-2-ethylindolizin-1-yl)glyoxylamide,
(DD) (8-(Carboxymethyloxy)-2-ethyl-3-(m-trifluoromethylbenzyl)indolizin-1-yl)glyoxylamide,
(EE) (8-Carboxymethyloxy-2-ethyl-3-(1-naphthylmethyl)indolizin-1-yl)glyoxylamide,
(FF) (8-(Carboxymethyloxy)-2-cyclopropyl-3-(o-phenylbenzyl)indolizin-1-yl) glyoxylamide,
(GG) mixtures of (Q) through (FF),
(HH) [[3-(2-Amino-1,2-dioxoethyl)-6-carboxyl-2-ethyl-1-benzyl-1H-indol-4-yl]oxy]acetic acid,
(II) [[3-(2-Amino-1,2-dioxoethyl)-6-methoxycarbonyl-2-ethyl-1-benzyl-1H-indol-4-yl]oxy]acetic acid,
(JJ) [[3-(2-Amino-1,2-dioxoethyl)-6-ethoxycarbonyl-2-ethyl-1-benzyl-1H-indol-4-yl]oxy]acetic acid,
(KK) [[3-(2-Amino-1,2-dioxoethyl)-6-n-propoxycarbonyl-2-ethyl 1-benzyl-1H-indol-4-yl]oxy]acetic acid,
(LL) [[3-(2-Amino-1,2-dioxoethyl)-6-i-propoxycarbonyl-2-ethyl-1-benzyl-1H-indol-4-yl]oxy]acetic acid,
(MM) [[3-(2-Amino-1,2-dioxoethyl)-6-cyclopropyloxycarbonyl-2-ethyl-1-benzyl-1H-indol-4-yl]oxy]acetic acid,
(NN) mixtures of (HH) through (MM).

6. A method of treatment of a mammal currently afflicted with disorders associated with apoptosis or previously afflicted with disorders associated with apoptosis, said method comprising administering to said mammal in need of such treatment a therapeutically effective amount of an N-heterocyclic glyoxylamide compound or a pharmaceutically acceptable salt, solvate, or a prodrug derivative thereof selected from the group consisting of compounds (I) and (I′):
(I) [[3-(2-Amino-1,2-dioxoethyl)-2-ethyl-1-(phenylmethyl)-1H-indol-4-yl]oxy]acetic acid,
(I′) [[3-(2-Amino-1,2-dioxoethyl)-2-ethyl-1-(phenylmethyl)-1H-indol-4-yl]oxy]acetic acid methyl ester.

7. A method of treatment of a mammal currently afflicted with disorders associated with apoptosis or previously afflicted with disorders associated with apoptosis, said method comprising administering to said mammal in need of such treatment a therapeutically effective amount of an N-heterocyclic glyoxylamide compound or a pharmaceutically acceptable salt, solvate, or a prodrug derivative thereof selected from the group consisting of compounds (A), (D), (H), (J) and (K):
(A) [[3-(2-Amino-1,2-dioxoethyl)-2-methyl-1-(phenylmethyl)-1H-indol-4-yl]oxy]acetic acid,
(D) [[3-(2-Amino-1,2-dioxoethyl)-1-([1,1′-biphenyl]-3-ylmethyl)-2-methyl-1H-indol-4-yl]oxy]acetic acid,
(H) [[3-(2-Amino-1,2-dioxoethyl)-2-methyl-1-[(1-naphthalenyl)methyl]-1H-indol-4-yl]oxy]acetic acid,
(J) [[3-(2-Amino-1,2-dioxoethyl)-1-[(3-chlorophenyl)methyl]-2-ethyl-1H-indol-4-yl]oxy]acetic acid,
(K) [[3-(2-Amino-1,2-dioxoethyl)-1-([1,1′-biphenyl]-2-ylmethyl)-2-ethyl-1H-indol-4-yl]oxy]acetic acid.

8. A method of treatment of a mammal currently afflicted with disorders associated with apoptosis or previously afflicted with disorders associated with apoptosis, said method comprising administering to said mammal in need of such treatment a therapeutically effective amount of an N-heterocyclic glyoxylamide compound or a pharmaceutically acceptable salt, solvate, or a prodrug derivative thereof selected from the group consisting of compounds (AA) and (AA′):
(AA) (8-(Carboxymethyloxy)-2-ethyl-3-(o-phenylbenzyl)indolizin-1-yl)glyoxylamide,
(AA′) (8-Carbomethoxymethyloxy)-2-ethyl-3-(o-phenylbenzyl)indolizin-1-yl) glyoxylamide.

9. A method of treatment of a mammal currently afflicted with disorders associated with apoptosis or previously afflicted with disorders associated with apoptosis, said method comprising administering to said mammal in need of such treatment a therapeutically effective amount of an N-heterocyclic glyoxylamide compound or a pharmaceutically acceptable salt, solvate, or a prodrug derivative thereof selected from the group consisting of compounds (HH) and (II):
(HH) [[3-(2-Amino-1,2-dioxoethyl)-6-carboxyl-2-ethyl-1-benzyl-1H-indol-4-yl]oxy]acetic acid,
(II) [[3-(2-Amino-1,2-dioxoethyl)-6-methoxycarbonyl-2-ethyl-1-benzyl-1H-indol-4-yl]oxy]acetic acid.

10. A method of treatment of a mammal currently afflicted with disorders associated with apoptosis or previously afflicted with disorders associated with apoptosis, said method comprising administering to said mammal in need of such treatment a therapeutically effective amount of an N-heterocyclic glyoxylamide compound selected from the formula: 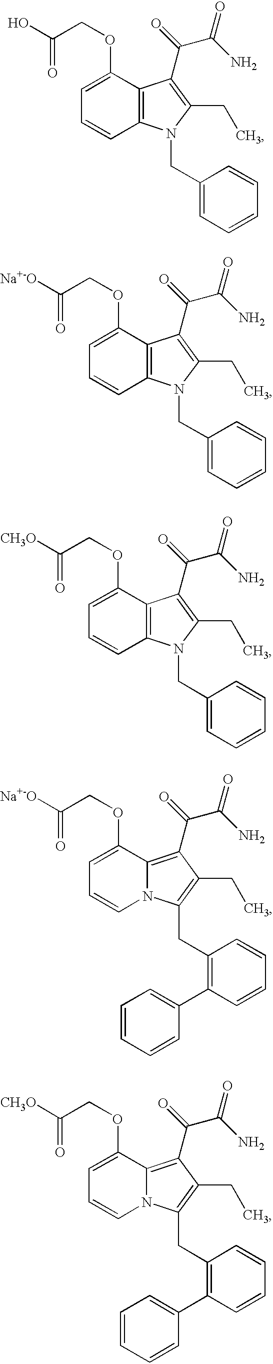 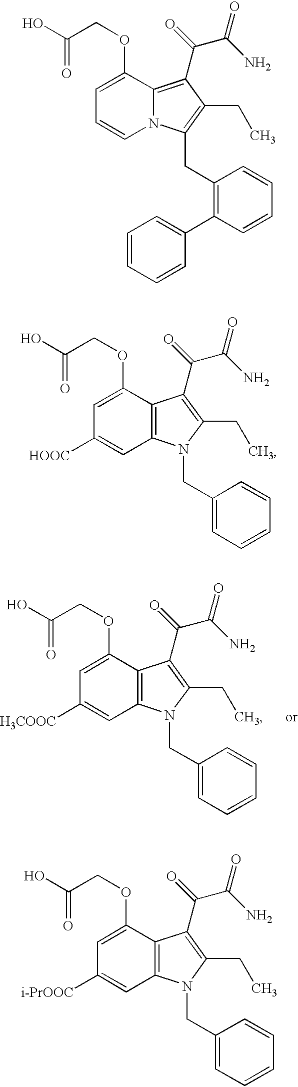
or a pharmaceutically acceptable salt, solvate, or a prodrug derivative thereof.

11. The method of claims 1 or 2 or 3 or 4 or 5 or 6 wherein the therapeutically effective amount of the compound is in the form of a pharmaceutical formulation comprising the compound and a suitable carrier or excipient therefor.

12. Use of an N-heterocyclic glyoxylamide compound for the manufacture of a medicant for treating disorders associated with apoptosis in a mammal, including a human, currently afflicted with disorders associated with apoptosis or previously afflicted with disorders associated with apoptosis;
where the compound is represented by the formula (I) or a pharmaceutically acceptable salt, solvate, or prodrug derivative thereof: 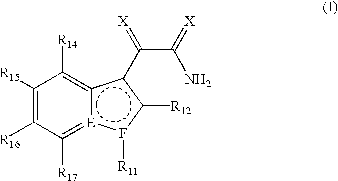
wherein
E and F are differently C or N;
--- is presence or absence of a double bond;
each X is independently oxygen or sulfur;
R₁₁ is selected from groups (a), (b) and (c) where;
(a) is C₇-C₂₀ alkyl, C₇-C₂₀ alkenyl, C₇-C₂₀ alkynyl; or carbocyclic radical selected from the group cycloalkyl, cycloalkenyl, phenyl, naphthyl, norbornanyl, bicycloheptadienyl, tolyl, xylyl, indenyl, stilbenyl, terphenylyl, diphenylethylenyl, phenyl-cyclohexenyl, acenaphthylenyl, and anthryl, biphenyl, bibenzylyl and related bibenzylyl homologues represented by the formula (bb), 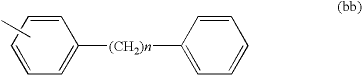
where
n is a number from 1 to 8; or
(b) is a member of (a) substituted with one or more independently selected non-interfering substituents selected from the group consisting of C₁-C₆ alkyl, C₂-C₆ alkenyl, C₂-C₆ alkynyl, C₇-C₁₂ aralkyl, C₇-C₁₂ alkaryl, C₃-C₈ cycloalkyl, C₃-C₈ cycloalkenyl, phenyl, tolyl, xylyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₁-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ—O—(C₁-C₆ alkyl), benzyloxy, phenoxy, phenylthio, —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl; where n is from 1 to 8;
(c) is the group -(L₁)-R₈₁; where, -(L₁)- is a divalent linking group having the formula; 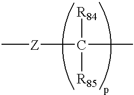
where,
R₈₄ and R₈₅ are each independently selected from hydrogen, C₁-C₁₀ alkyl, carboxyl, carbalkoxy, or halo;
p is 1 to 5,
Z is a bond, —(CH₂)—, —O—, —N(C₁-C₁₀ alkyl)-, —NH—, or —S—; and where R₈₁ is a group selected from (a) or (b);
R₁₂ is hydrogen, halo, C₁-C₃ alkyl, C₃-C₄ cycloalkyl, C₃-C₄ cycloalkenyl, —O—(C₁-C₂ alkyl), or —S—(C₁-C₂ alkyl);
R₁₄ is hydrogen or a group, -(Lₐ)-(acidic group) wherein -(Lₐ)- is represented by the formula; 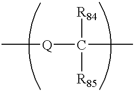
where
Q is selected from the group —(CH₂)—, —O—, —NH—, and —S—, and R₈₄ and R₈₅ are each independently selected from hydrogen, C₁-C₁₀ alkyl, aryl, C₁-C₁₀ alkaryl, C₁-C₁₀ aralkyl, and halo;
R₁₅ is hydrogen or a group, -(L_{a′})-(acidic group) wherein -(L_{a′})- is represented by the formula; 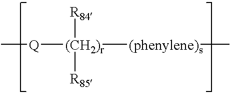
where
r is a number from 1 to 7, s is 0 or 1, and Q is selected from the group —(CH₂)—, —O—, —NH—, and —S—, and R_{84′} and R_{85′} are each independently selected from hydrogen, C₁-C₁₀ alkyl, aryl, C₁-C₁₀ alkaryl, C₁-C₁₀ aralkyl, carboxy, carbalkoxy, and halo; provided that at least one of R₁₄ or R₁₅ must be the group, -(La)-(acidic group) or -(La′)-(acidic group);
R₁₆ Is hydrogen, carboxyl or ester thereof;
R₁₇ is selected from hydrogen, non-interfering substituents, selected from the group consisting of C₁-C₆ alkyl, C₂-C₆ alkenyl, C₂-C₆ alkynyl, C₇-C₁₂ aralkyl, C₇-C₁₂ alkaryl, C₃-C₈ cycloalkyl, C₃-C₈ cycloalkenyl, phenyl, tolyl, xylyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ—O—(C₁-C₆ alkyl), benzyloxy, phenoxy, phenylthio, —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl; where n is from 1 to 8.

13. Use of a 1H-indole-3-glyoxylamide compound for the manufacture of a medicant for treating disorders associated with apoptosis in a mammal, including a human, currently afflicted with disorders associated with apoptosis or previously afflicted with disorders associated with apoptosis;
where the compound is represented by the formula (II) or a pharmaceutically acceptable salt, solvate, or prodrug derivative thereof: 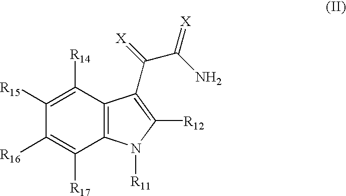
wherein
X, R₁₁, R₁₂, R₁₄, R₁₅, R₁₆ and R₁₇ are as defined above.

14. Use of an indolizine-1-glyoxylamide compound for the manufacture of a medicant for treating disorders associated with apoptosis in a mammal, including a human, currently afflicted with disorders associated with apoptosis or previously afflicted with disorders associated with apoptosis;
where the compound is represented by the formula (III) or a pharmaceutically acceptable salt, solvate, or prodrug derivative thereof: 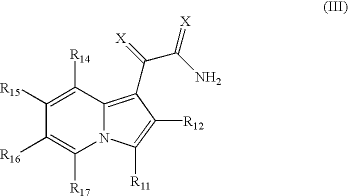
wherein
X, R₁₁, R₁₂, R₁₄, R₁₅, R₁₆ and R₁₇ are as defined above.

15. Use of an N-heterocyclic glyoxylamide compound for the manufacture of a medicant for treating disorders associated with apoptosis in a mammal, including a human, currently afflicted with disorders associated with apoptosis or previously afflicted with disorders associated with apoptosis;
where the compound is an N-heterocyclic glyoxylamide compound or a pharmaceutically acceptable salt, solvate, or prodrug derivative thereof selected from the group consisting of compounds (A) through (NN):
(A) [[3-(2-Amino-1,2-dioxoethyl)-2-methyl-1-(phenylmethyl)-1H-indol-4-yl]oxy]acetic acid,
(B) dl-2-[[3-(2-Amino-1,2-dioxoethyl)-2-methyl-1-(phenylmethyl)-1H-indol-4-yl]oxy]propanoic acid,
(C) [[3-(2-Amino-1,2-dioxoethyl)-1-([1,1′-biphenyl]-2-ylmethyl)-2-methyl-1H-indol-4-yl]oxy]acetic acid,
(D) [[3-(2-Amino-1,2-dioxoethyl)-1-([1,1′-biphenyl]-3-ylmethyl)-2-methyl-1H-indol-4-yl]oxy]acetic acid,
(E) [[3-(2-Amino-1,2-dioxoethyl)-1-([1,1′-biphenyl]-4-ylmethyl)-2-methyl-1H-indol-4-yl]oxy]acetic acid,
(F) [[3-(2-Amino-1,2-dioxoethyl)-1-[(2,6-dichlorophenyl)methyl]-2-methyl-1H-indol-4-yl]oxy]acetic acid,
(G) [[3-(2-Amino-1,2-dioxoethyl)-1-[(4-fluorophenyl)methyl]-2-methyl-1H-indol-4-yl]oxy]acetic acid,
(H) [[3-(2-Amino-1,2-dioxoethyl)-2-methyl-1-[(1-naphthalenyl)methyl]-1H-indol-4-yl]oxy]acetic acid,
(I) [[3-(2-Amino-1,2-dioxoethyl)-2-ethyl-1-(phenylmethyl)-1H-indol-4-yl]oxy]acetic acid,
(I′) [[3-(2-Amino-1,2-dioxoethyl)-2-ethyl-1-(phenylmethyl)-1H-indol-4-yl]oxy]acetic acid methyl ester
(J) [[3-(2-Amino-1,2-dioxoethyl)-1-[(3-chlorophenyl)methyl]-2-ethyl-1H-indol-4-yl]oxy]acetic acid,
(K) [[3-(2-Amino-1,2-dioxoethyl)-1-([1,1′-biphenyl]-2-ylmethyl)-2-ethyl-1H-indol-4-yl]oxy]acetic acid,
(L) [[3-(2-amino-1,2-dioxoethyl)-1-([1,1′-biphenyl]-2-ylmethyl)-2-propyl-1H-indol-4-yl]oxy]acetic acid,
(M) [[3-(2-Amino-1,2-dioxoethyl)-2-cyclopropyl-1-(phenylmethyl)-1H-indol-4-yl]oxy]acetic acid,
(N) [[3-(2-Amino-1,2-dioxoethyl)-1-([1,1′-biphenyl]-2-ylmethyl)-2-cyclopropyl-1H-indol-4-yl]oxy]acetic acid,
(O) 4-[[3-(2-Amino-1,2-dioxoethyl)-2-ethyl-1-(phenylmethyl)-1H-indol-5-yl]oxy]butanoic acid,
(P) mixtures of (A) through (O),
(Q) (8-(Carbomethoxymethyloxy)-2-ethyl-3-(o-phenylbenzyl)indolizin-1-yl)glyoxylamide,
(R) (3-Benzyl-8-(carbethoxymethyloxy)-2-ethylindolizin-1-yl)glyoxylamide,
(S) (8-(Carbethoxymethyloxy)-2-ethyl-3-(o-phenylbenzyl)indolizin-1-yl)glyoxylamide,
(T) (3-Benzyl-8-(carbethoxymethyloxy)-2-methylindolizin-1-yl)glyoxylamide,
(U) (8-(Carbethoxymethyloxy)-3-(m-chlorobenzyl)-2-ethylindolizin-1-yl)glyoxylamide,
(V) (8-Carbethoxymethyloxy-2-ethyl-3-(1-naphthylmethyl)indolizin-1-yl)glyoxylamide,
(W) (3-Benzyl-8-(t-butoxycarbonylmethyloxy)-2-ethylindolizin-1-yl)glyoxylamide,
(X) (8-(Carbmethoxymethyloxy)-2-ethyl-3-(m-trifluoromethylbenzyl)indolizin-1-yl)glyoxylamide,
(Y) (8-(Carbmethoxymethyloxy)-2-cyclopropyl-3-(o-phenylbenzyl)indolizin-1-yl)glyoxylamide,
(Z) (3-Benzyl-8-(carboxymethyloxy)-2-ethylindolizin-1-yl)glyoxylamide,
(AA) (8-(Carboxymethyloxy)-2-ethyl-3-(o-phenylbenzyl)indolizin-1-yl)glyoxylamide,
(AA′) (8-Carbomethoxymethyloxy)-2-ethyl-3-(o-phenylbenzyl)indolizin-1-yl)glyoxylamide,
(BB) (3-Benzyl-8-(carboxymethyloxy)-2-methylindolizin-1-yl)glyoxylamide,
(CC) (8-(Carboxymethyloxy)-3-(m-chlorobenzyl)-2-ethylindolizin-1-yl)glyoxylamide,
(DD) (8-(Carboxymethyloxy)-2-ethyl-3-(m-trifluoromethylbenzyl)indolizin-1-yl)glyoxylamide,
(EE) (8-Carboxymethyloxy-2-ethyl-3-(1-naphthylmethyl)indolizin-1-yl)glyoxylamide,
(FF) (8-(Carboxymethyloxy)-2-cyclopropyl-3-(o-phenylbenzyl)indolizin-1-yl) glyoxylamide,
(GG) mixtures of (Q) through (FF),
(HH) [[3-(2-Amino-1,2-dioxoethyl)-6-carboxyl-2-ethyl-1-benzyl-1H-indol-4-yl]oxy]acetic acid,
(II) [[3-(2-Amino-1,2-dioxoethyl)-6-methoxycarbonyl-2-ethyl-1-benzyl-1H-indol-4-yl]oxy]acetic acid,
(JJ) [[3-(2-Amino-1,2-dioxoethyl)-6-ethoxycarbonyl-2-ethyl-1-benzyl-1H-indol-4-yl]oxy]acetic acid,
(KK) [[3-(2-Amino-1,2-dioxoethyl)-6-n-propoxycarbonyl-2-ethyl-1-benzyl-1H-indol-4-yl]oxy]acetic acid,
(LL) [[3-(2-Amino-1,2-dioxoethyl)-6-i-propoxycarbonyl-2-ethyl-1-benzyl-1H-indol-4-yl]oxy]acetic acid,
(MM) [[3-(2-Amino-1,2-dioxoethyl)-6-cyclopropyloxycarbonyl-2-ethyl-1-benzyl-1H-indol-4-yl]oxy]acetic acid,
(NN) mixtures of (HH) through (MM).

16. Use of an N-heterocyclic glyoxylamide compound for the manufacture of a medicant for treating disorders associated with apoptosis in a mammal, including a human, currently afflicted with disorders associated with apoptosis or previously afflicted with disorders associated with apoptosis;
where the compound is an N-heterocyclic glyoxylamide compound or a pharmaceutically acceptable salt, solvate, or prodrug derivative thereof selected from the group consisting of compounds (I) and (I′):
(I) [[3-(2-Amino-1,2-dioxoethyl)-2-ethyl-1-(phenylmethyl)-1H-indol-4-yl]oxy]acetic acid,
(I′) [[3-(2-Amino-1,2-dioxoethyl)-2-ethyl-1-(phenylmethyl)-1H-indol-4-yl]oxy]acetic acid methyl ester.

17. Use of an N-heterocyclic glyoxylamide compound for the manufacture of a medicant for treating disorders associated with apoptosis in a mammal, including a human, currently afflicted with disorders associated with apoptosis or previously afflicted with disorders associated with apoptosis;
where the compound is an N-heterocyclic glyoxylamide compound or a pharmaceutically acceptable salt, solvate, or prodrug derivative thereof selected from the group consisting of compounds (A), (D), (H), (J) and (K):
(A) [[3-(2-Amino-1,2-dioxoethyl)-2-methyl-1-(phenylmethyl)-1H -indol-4-yl]oxy]acetic acid,
(D) [[3-(2-Amino-1,2-dioxoethyl)-1-([1,1′-biphenyl]-3-ylmethyl)-2-methyl-1H-indol-4-yl]oxy]acetic acid,
(H) [[3-(2-Amino-1,2-dioxoethyl)-2-methyl-1-[(1-naphthalenyl)methyl]-1H-indol-4-yl]oxy]acetic acid,
(J) [[3-(2-Amino-1,2-dioxoethyl)-1-[(3-chlorophenyl)methyl]-2-ethyl-1H-indol-4-yl]oxy]acetic acid,
(K) [[3-(2-Amino-1,2-dioxoethyl)-1-([1,1′-biphenyl]-2-ylmethyl)-2-ethyl-1H-indol-4-yl]oxy]acetic acid.

18. Use of an N-heterocyclic glyoxylamide compound for the manufacture of a medicant for treating disorders associated with apoptosis in a mammal, including a human, currently afflicted with disorders associated with apoptosis or previously afflicted with disorders associated with apoptosis;
where the compound is an N-heterocyclic glyoxylamide compound or a pharmaceutically acceptable salt, solvate, or prodrug derivative thereof selected from the group consisting of compounds (AA) and (AA′):
(AA) (8-(Carboxymethyloxy)-2-ethyl-3-(o-phenylbenzyl)indolizin-1-yl)glyoxylamide,
(AA′) (8-Carbomethoxymethyloxy)-2-ethyl-3-(o-phenylbenzyl)indolizin-1-yl)glyoxylamide.

19. Use of an N-heterocyclic glyoxylamide compound for the manufacture of a medicant for treating disorders associated with apoptosis in a mammal, including a human, currently afflicted with disorders associated with apoptosis or previously afflicted with disorders associated with apoptosis;
where the compound is an N-heterocyclic glyoxylamide compound or a pharmaceutically acceptable salt, solvate, or prodrug derivative thereof selected from the group consisting of compounds (HH) and (II):
(HH) [[3-(2-Amino-1,2-dioxoethyl)-6-carboxyl-2-ethyl-1-benzyl-1H-indol-4-yl]oxy]acetic acid,
(II) [[3-(2-Amino-1, 2-dioxoethyl)-6-methoxycarbonyl-2-ethyl-1-benzyl-1H-indol-4-yl]oxy]acetic acid.

20. Use of an N-heterocyclic glyoxylamide compound for the manufacture of a medicant for treating disorders associated with apoptosis in a mammal, including a human, currently afflicted with disorders associated with apoptosis or previously afflicted with disorders associated with apoptosis;
where the compound is an N-heterocyclic glyoxylamide compound selected from the formula: 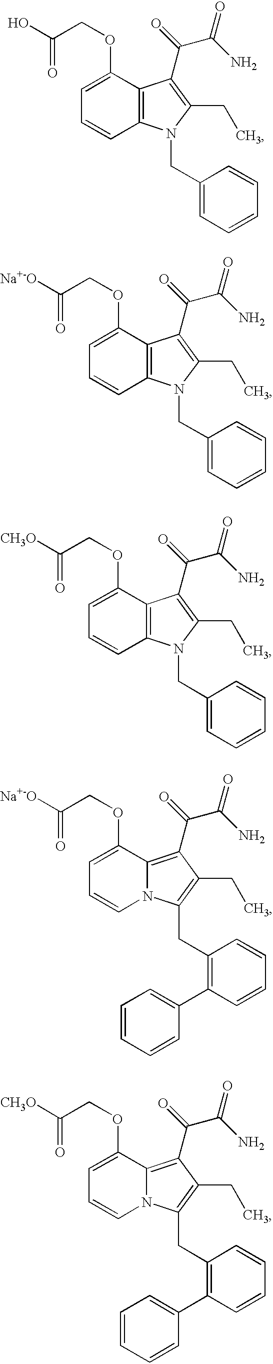 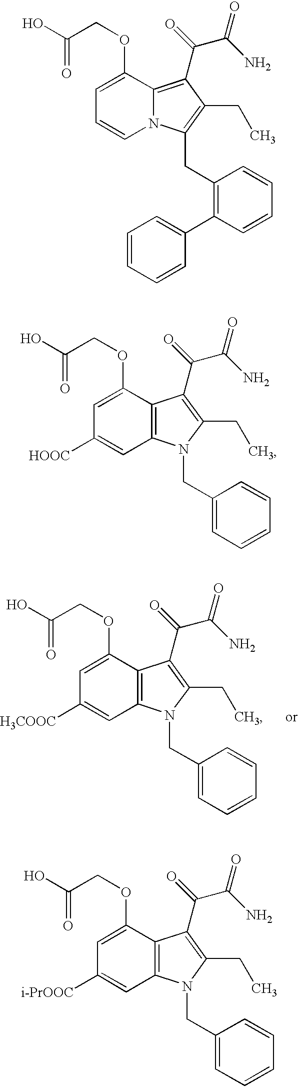
or a pharmaceutically acceptable salt, solvate, or a prodrug derivative thereof.

21. A composition for treatment of disorders associated with apoptosis;
which comprises an N-heterocyclic glyoxylamide compound represented by the formula (I) or a pharmaceutically acceptable salt, solvate, or prodrug derivative thereof: 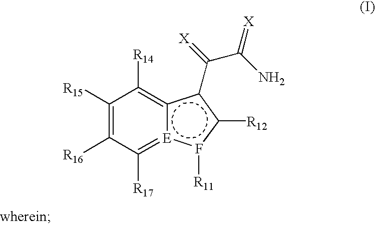
E and F are differently C or N;
--- is presence or absence of a double bond;
each X is independently oxygen or sulfur;
R₁₁ is selected from groups (a), (b) and (c) where;
(a) is C₇-C₂₀ alkyl, C₇-C₂₀ alkenyl, C₇-C₂₀ alkynyl; or carbocyclic radical selected from the group cycloalkyl, cycloalkenyl, phenyl, naphthyl, norbornanyl, bicycloheptadienyl, tolyl, xylyl, indenyl, stilbenyl, terphenylyl, diphenylethylenyl, phenyl-cyclohexenyl, acenaphthylenyl, and anthryl, biphenyl, bibenzylyl and related bibenzylyl homologues represented by the formula (bb), 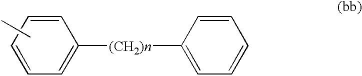
where
n is a number from 1 to 8; or
(b) is a member of (a) substituted with one or more independently selected non-interfering substituents selected from the group consisting of C₁-C₆ alkyl, C₂-C₆ alkenyl, C₂-C₆ alkynyl, C₇-C₁₂ aralkyl, C₇-C₁₂ alkaryl, C₃-C₈ cycloalkyl, C₃-C₈ cycloalkenyl, phenyl, tolyl, xylyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₁-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ—O—(C₁-C₆ alkyl), benzyloxy, phenoxy, phenylthio, —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl; where n is from 1 to 8;
(c) is the group -(L₁)-R₈₁; where, -(L₁)- is a divalent linking group having the formula; 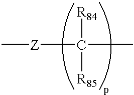
where,
R₈₄ and R₈₅ are each independently selected from hydrogen, C₁-C₁₀ alkyl, carboxyl, carbalkoxy, or halo;
p is 1 to 5,
Z is a bond, —(CH₂)—, —O—, —N(C₁-C₁₀ alkyl)-, —NH—, or —S—; and where R₈₁ is a group selected from (a) or (b);
R₁₂ is hydrogen, halo, C₁-C₃ alkyl, C₃-C₄ cycloalkyl, C₃-C₄ cycloalkenyl, —O—(C₁-C₂ alkyl), or —S—(C₁-C₂ alkyl);
R₁₄ is hydrogen or a group, -(Lₐ)-(acidic group) wherein -(Lₐ)- is represented by the formula; 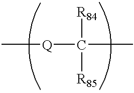
where
Q is selected from the group —(CH₂)—, —O—, —NH—, and —S—, and R₈₄ and R₈₅ are each independently selected from hydrogen, C₁-C₁₀ alkyl, aryl, C₁-C₁₀ alkaryl, C₁-C₁₀ aralkyl, and halo;
R₁₅ is hydrogen or a group, -(La′)-(acidic group) wherein -(La′)- is represented by the formula; 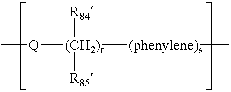
where
r is a number from 1 to 7, s is 0 or 1, and Q is selected from the group —(CH₂)—, —O—, —NH—, and —S—, and R_{84′} and R_{85′} are each independently selected from hydrogen, C₁-C₁₀ alkyl, aryl, C₁-C₁₀ alkaryl, C₁-C₁₀ aralkyl, carboxy, carbalkoxy, and halo; provided that at least one of R14 or R15 must be the group, -(La)-(acidic group) or -(La′)-(acidic group);
R16 is hydrogen, carboxyl or ester thereof;
R17 is selected from hydrogen, non-interfering substituents, selected from the group consisting of C₁-C₆ alkyl, C₂-C₆ alkenyl, C₂-C₆ alkynyl, C₇-C₁₂ aralkyl, C₇-C₁₂ alkaryl, C₃-C₈ cycloalkyl, C₃-C₈ cycloalkenyl, phenyl, tolyl, xylyl, biphenyl, C₁-C₆ alkoxy, C₂-C₆ alkenyloxy, C₂-C₆ alkynyloxy, C₂-C₁₂ alkoxyalkyl, C₂-C₁₂ alkoxyalkyloxy, C₂-C₁₂ alkylcarbonyl, C₂-C₁₂ alkylcarbonylamino, C₂-C₁₂ alkoxyamino, C₂-C₁₂ alkoxyaminocarbonyl, C₂-C₁₂ alkylamino, C₁-C₆ alkylthio, C₂-C₁₂ alkylthiocarbonyl, C₁-C₆ alkylsulfinyl, C₁-C₆ alkylsulfonyl, C₂-C₆ haloalkoxy, C₁-C₆ haloalkylsulfonyl, C₂-C₆ haloalkyl, C₁-C₆ hydroxyalkyl, —C(O)O(C₁-C₆ alkyl), —(CH₂)ₙ—O—(C₁-C₆ alkyl), benzyloxy, phenoxy, phenylthio, —CHO, amino, amidino, bromo, carbamyl, carboxyl, carbalkoxy, —(CH₂)ₙ—CO₂H, chloro, cyano, cyanoguanidinyl, fluoro, guanidino, hydrazide, hydrazino, hydrazido, hydroxy, hydroxyamino, iodo, nitro, phosphono, —SO₃H, thioacetal, thiocarbonyl, and C₁-C₆ carbonyl; where n is from 1 to 8.

22. A composition for treatment of disorders associated with apoptosis;
which comprises a IH-indole-3-glyoxylamide compound represented by the formula (II) or a pharmaceutically acceptable salt, solvate, or prodrug derivative thereof: 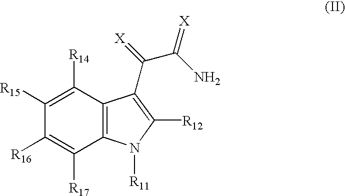
wherein
X, R₁₁, R₁₂, R₁₄, R₁₅, R₁₆ and R₁₇ are as defined above.

23. A composition for treatment of disorders associated with apoptosis;
which comprises an indolizine-1-glyoxylamide compound represented by the formula (III) or a pharmaceutically acceptable salt, solvate, or prodrug derivative thereof: 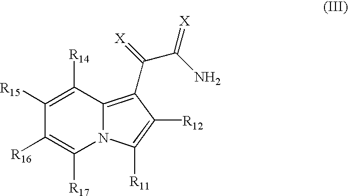
wherein
X, R₁₁, R₁₂, R₁₄, R₁₅, R₁₆ and R₁₇ are as defined above.

24. A composition for treatment of disorders associated with apoptosis;
which comprises an N-heterocyclic glyoxylamide compound or a pharmaceutically acceptable salt, solvate, or prodrug derivative thereof selected from the group consisting of compounds (A) through (NN):
(A) [[3-(2-Amino-1,2-dioxoethyl)-2-methyl-1-(phenylmethyl)-1H-indol-4-yl]oxy]acetic acid,
(B) dl-2-[[3-(2-Amino-1,2-dioxoethyl)-2-methyl-1-(phenylmethyl)-1H-indol-4-yl]oxy]propanoic acid,
(C) [[3-(2-Amino-1,2-dioxoethyl)-1-([1,1′-biphenyl]-2-ylmethyl)-2-methyl-1H-indol-4-yl]oxy]acetic acid,
(D) [[3-(2-Amino-1,2-dioxoethyl)-1-([1,1′-biphenyl]-3-ylmethyl)-2-methyl-1H-indol-4-yl]oxy]acetic acid,
(E) [[3-(2-Amino-1,2-dioxoethyl)-1-([1,1′-biphenyl]-4-ylmethyl)-2-methyl-1H-indol-4-yl]oxy]acetic acid,
(F) [[3-(2-Amino-1,2-dioxoethyl)-1-[(2,6-dichlorophenyl)methyl]-2-methyl-1H-indol-4-yl]oxy]acetic acid,
(G) [[3-(2-Amino-1,2-dioxoethyl)-1-[(4-fluorophenyl)methyl]-2-methyl-1H-indol-4-yl]oxy]acetic acid,
(H) [[3-(2-Amino-1,2-dioxoethyl)-2-methyl-1-[(1-naphthalenyl)methyl]-1H-indol-4-yl]oxy]acetic acid,
(I) [[3-(2-Amino-1,2-dioxoethyl)-2-ethyl-1-(phenylmethyl)-1H-indol-4-yl]oxy]acetic acid,
(I′) [[3-(2-Amino-1,2-dioxoethyl)-2-ethyl-1-(phenylmethyl)-1H-indol-4-yl]oxy]acetic acid methyl ester
(J) [[3-(2-Amino-1,2-dioxoethyl)-1-[(3-chlorophenyl)methyl]-2-ethyl-1H-indol-4-yl]oxy]acetic acid,
(K) [[3-(2-Amino-1,2-dioxoethyl)-1-([1,1′-biphenyl]-2-ylmethyl)-2-ethyl-1H-indol-4-yl]oxy]acetic acid,
(L) [[3-(2-amino-1,2-dioxoethyl)-1-([1,1′-biphenyl]-2-ylmethyl)-2-propyl-1H-indol-4-yl]oxy]acetic acid,
(M) [[3-(2-Amino-1,2-dioxoethyl)-2-cyclopropyl-1-(phenylmethyl)-1H-indol-4-yl]oxy]acetic acid,
(N) [[3-(2-Amino-1,2-dioxoethyl)-1-([1,1′-biphenyl]-2-ylmethyl)-2-cyclopropyl-1H-indol-4-yl]oxy]acetic acid,
(O) 4-[[3-(2-Amino-1,2-dioxoethyl)-2-ethyl-1-(phenylmethyl)-1H-indol-5-yl]oxy]butanoic acid,
(P) mixtures of (A) through (O),
(Q) (8-(Carbomethoxymethyloxy)-2-ethyl-3-(o-phenylbenzyl)indolizin-1-yl)glyoxylamide,
(R) (3-Benzyl-8-(carbethoxymethyloxy)-2-ethylindolizin-1-yl)glyoxylamide,
(S) (8-(Carbethoxymethyloxy)-2-ethyl-3-(o-phenylbenzyl)indolizin-1-yl)glyoxylamide,
(T) (3-Benzyl-8-(carbethoxymethyloxy)-2-methylindolizin-1-yl)glyoxylamide,
(U) (8-(Carbethoxymethyloxy)-3-(m-chlorobenzyl)-2-ethylindolizin-1-yl)glyoxylamide,
(V) (8-Carbethoxymethyloxy-2-ethyl-3-(1-naphthylmethyl)indolizin-1-yl)glyoxylamide,
(W) (3-Benzyl-8-(t-butoxycarbonylmethyloxy)-2-ethylindolizin-1-yl)glyoxylamide,
(X) (8-(Carbmethoxymethyloxy)-2-ethyl-3-(m-trifluoromethylbenzyl)indolizin-1-yl)glyoxylamide,
(Y) (8-(Carbmethoxymethyloxy)-2-cyclopropyl-3-(o-phenylbenzyl)indolizin-1-yl)glyoxylamide,
(Z) (3-Benzyl-8-(carboxymethyloxy)-2-ethylindolizin-1-yl)glyoxylamide,
(AA) (8-(Carboxymethyloxy)-2-ethyl-3-(o-phenylbenzyl)indolizin-1-yl)glyoxylamide,
(AA′) (8-Carbomethoxymethyloxy)-2-ethyl-3-(o-phenylbenzyl)indolizin-1-yl)glyoxylamide,
(BB) (3-Benzyl-8-(carboxymethyloxy)-2-methylindolizin-1-yl)glyoxylamide,
(CC) (8-(Carboxymethyloxy)-3-(m-chlorobenzyl)-2-ethylindolizin-1-yl)glyoxylamide,
(DD) (8-(Carboxymethyloxy)-2-ethyl-3-(m-trifluoromethylbenzyl)indolizin-1-yl)glyoxylamide,
(EE) (8-Carboxymethyloxy-2-ethyl-3-(1-naphthylmethyl)indolizin-1-yl)glyoxylamide,
(FF) (8-(Carboxymethyloxy)-2-cyclopropyl-3-(o-phenylbenzyl)indolizin-1-yl) glyoxylamide,
(GG) mixtures of (Q) through (FF),
(HH) [[3-(2-Amino-1,2-dioxoethyl)-6-carboxyl-2-ethyl-1-benzyl-1H-indol-4-yl]oxy]acetic acid,
(II) [[3-(2-Amino-1,2-dioxoethyl)-6-methoxycarbonyl-2-ethyl-1-benzyl-1H-indol-4-yl]oxy]acetic acid,
(JJ) [[3-(2-Amino-1,2-dioxoethyl)-6-ethoxycarbonyl-2-ethyl-1-benzyl-1H-indol-4-yl]oxy]acetic acid,
(KK) [[3-(2-Amino-1,2-dioxoethyl)-6-n-propoxycarbonyl-2-ethyl 1-benzyl-1H-indol-4-yl]oxy]acetic acid,
(LL) [[3-(2-Amino-1,2-dioxoethyl)-6-i-propoxycarbonyl-2-ethyl-1-benzyl-1H-indol-4-yl]oxy]acetic acid,
(MM) [[3-(2-Amino-1,2-dioxoethyl)-6-cyclopropyloxycarbonyl-2-ethyl-1-benzyl-1H-indol-4-yl]oxy]acetic acid,
(NN) mixtures of (HH) through (MM).

25. A composition for treatment of disorders associated with apoptosis;
which comprises an N-heterocyclic glyoxylamide compound or a pharmaceutically acceptable salt, solvate, or prodrug derivative thereof selected from the group consisting of compounds (I) and (I′):
(I) [[3-(2-Amino-1,2-dioxoethyl)-2-ethyl-1-(phenylmethyl)-1H-indol-4-yl]oxy]acetic acid,
(I′) [[3-(2-Amino-1,2-dioxoethyl)-2-ethyl-1-(phenylmethyl)-1H-indol-4-yl]oxy]acetic acid methyl ester.

26. A composition for treatment of disorders associated with apoptosis;
which comprises an N-heterocyclic glyoxylamide compound or a pharmaceutically acceptable salt, solvate, or prodrug derivative thereof selected from the group consisting of compounds (A), (D), (H), (J) and (K):
(A) [[3-(2-Amino-1,2-dioxoethyl)-2-methyl-1-(phenylmethyl)-1H-indol-4-yl]oxy]acetic acid,
(D) [[3-(2-Amino-1,2-dioxoethyl)-1-([1,1′-biphenyl]-3-ylmethyl)-2-methyl-1H-indol-4-yl]oxy]acetic acid,
(H) [[3-(2-Amino-1,2-dioxoethyl)-2-methyl-1-[(1-naphthalenyl)methyl]-1H-indol-4-yl]oxy]acetic acid,
(J) [[3-(2-Amino-1,2-dioxoethyl)-1-[(3-chlorophenyl)methyl]-2-ethyl-1H-indol-4-yl]oxy]acetic acid,
(K) [[3-(2-Amino-1,2-dioxoethyl)-1-([1,1′-biphenyl]-2-ylmethyl)-2-ethyl-1H-indol-4-yl]oxy]acetic acid.

27. A composition for treatment of disorders associated with apoptosis;
which comprises an N-heterocyclic glyoxylamide compound or a pharmaceutically acceptable salt, solvate, or prodrug derivative thereof selected from the group consisting of compounds (AA) and (AA′):
(AA) (8-(Carboxymethyloxy)-2-ethyl-3-(o-phenylbenzyl)indolizin-1-yl)glyoxylamide,
(AA′) (8-Carbomethoxymethyloxy)-2-ethyl-3-(o-phenylbenzyl)indolizin-1-yl)glyoxylamide.

28. A composition for treatment of disorders associated with apoptosis;
which comprises an N-heterocyclic glyoxylamide compound or a pharmaceutically acceptable salt, solvate, or prodrug derivative thereof selected from the group consisting of compounds (HH) and (II):
(HH) [[3-(2-Amino-1,2-dioxoethyl)-6-carboxyl-2-ethyl-1-benzyl-1H-indol-4-yl]oxy]acetic acid,
(II) [[3-(2-Amino-1,2-dioxoethyl) -6-methoxycarbonyl-2-ethyl-1-benzyl-1H-indol-4-yl]oxy]acetic acid.

29. A composition for treatment of disorders associated with apoptosis;
which comprises an N-heterocyclic glyoxylamide compound selected from the formula: 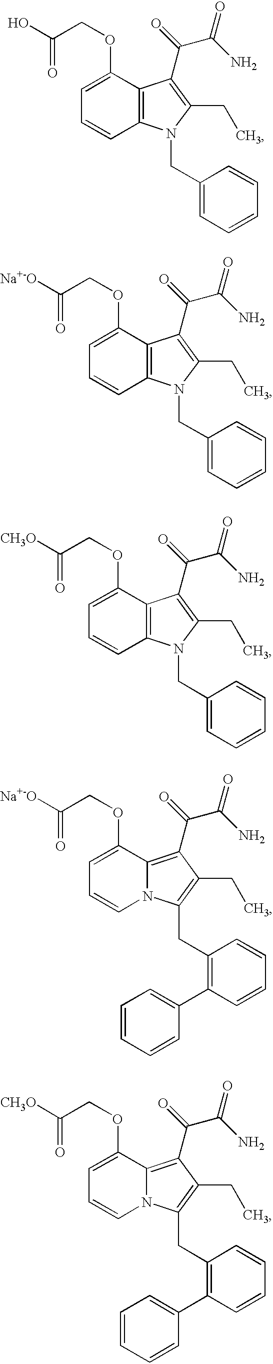 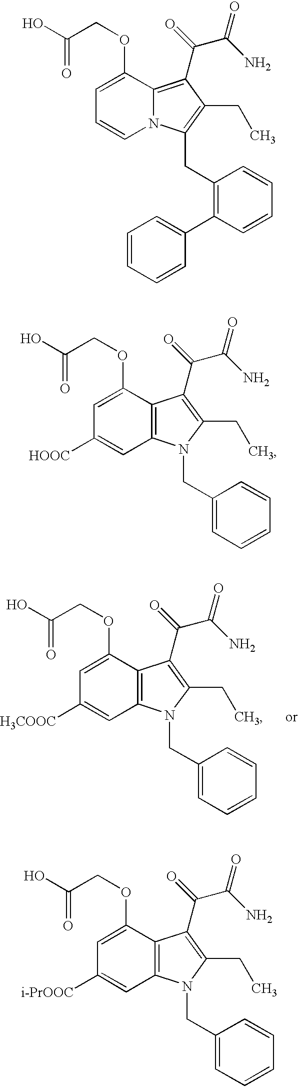
or a pharmaceutically acceptable salt, solvate, or a prodrug derivative thereof.
